(19) United States
(12) Patent Application Publication
(10) Pub. No.: US 2021/0322646 A1
(43) Pub. Date: Oct. 21, 2021

(54) THERMALLY RESPONSIVE SHAPE MEMORY POLYMER ACTUATOR, PROSTHESIS INCORPORATING SAME, AND FABRICATION METHOD

(71) Applicants: Steven Lathers, Englewood, CO (US); Jeffrey LaBelle, Tempe, AZ (US)

(72) Inventors: Steven Lathers, Englewood, CO (US); Jeffrey LaBelle, Tempe, AZ (US)

(73) Assignee: ARIZONA BOARD OF REGENTS ON BEHALF OF ARIZONA STATE UNIVERSITY, Scottsdale, AZ (US)

(21) Appl. No.: 17/364,256

(22) Filed: Jun. 30, 2021

Related U.S. Application Data
(63) Continuation of application No. 16/200,824, filed on Nov. 27, 2018.
(60) Provisional application No. 62/590,970, filed on Nov. 27, 2017.

(51) Int. Cl. A61L 27/50; F03G 7/06; A61L 27/26; A61F 2/68; A61F 2/60; A61F 2/50; C08L 37/00; C08L 23/00

(52) U.S. Cl. CPC A61L 27/50; F03G 7/065; A61L 27/26; A61F 2/68; A61F 2/604; A61F 2002/74; C08L 37/00; C08L 23/00; A61L 2400/16; A61F 2002/5056; A61F 2002/5066; A61F 2/50

(57) ABSTRACT

A thermally responsive shape memory polymer (SMP) actuator includes a body having at least one non-linear segment arranged between first and second ends, with the body comprising a plurality of dots, rods, or layers of SMP material. The SMP material my include a linear aliphatic thermoplastic polyester and at least one other polymer. The non-linear segment may have a substantially flat zig-zag shape arranged between first and second substantially straight segments. A prosthetic device may include multiple thermally responsive shape memory actuators and a movable joint arranged between structural members having anchors associated therewith. At least one first SMP actuator provides pivotal movement in a first direction, and at least one second SMP actuator provides pivotal movement in a second direction. Methods for forming SMP actuators include body formation by additive manufacturing, heating the body to a glass transition temperature range while applying tension, and cooling the body.

### STATEMENT OF RELATED APPLICATION(S)

This application is a continuation of U.S. patent application Ser. No. 16/200,824 filed on Nov. 27, 2018, which claims priority to U.S. Provisional Patent Application No. 62/590,970 filed on Nov. 27, 2017, wherein the entire contents of the foregoing applications are hereby incorporated by reference herein.

### TECHNICAL FIELD

This disclosure relates to shape memory polymer actuators, prosthetic devices incorporating shape memory polymer actuators, and methods for fabrication and use of shape memory polymer actuators.

### BACKGROUND

In the United States, about two million people have lost a limb, and about 185,000 people each year lose a limb, with hospital costs for amputations of approximately $8.3 billion each year. 54% of limb losses are attributable to vascular diseases, including diabetes and peripheral arterial disease; about 45% of limb losses are attributable to physical trauma; and fewer than 2% of limb losses are attributable to cancer, with a ratio of upper limb loss to lower limb loss of 1:4. Prosthetics can cost up to $50,000 per limb, and a significant number (possibly a majority) are not covered by insurance. Additionally, many prosthetics need to be replaced as the user grows, and health insurance frequently does not cover the cost of continual replacement and/or modification of prosthetics.

For upper limb amputations, conventional functional prosthetics include categories of body-powered systems and electric/intelligent systems. Body-powered prosthetics typically use cables and harnesses strapped to the individual to mechanically maneuver the artificial limb with the use of an intact anatomical system. Body-powered systems are lightweight, inexpensive, and lack complexity; however, such systems lack feedback, are unable to provide high force output, and can be fatiguing to operate. Conventional electric prosthetic systems use high powered direct current and/or servo motors in conjunction with a feedback/control system that collects input from electrodes monitoring muscular (EMG) activity or neural (EEG) activity. Downsides of electric systems are that they are expensive, heavy, and noisy. Regarding the weight issue, for example, EMG control hands can weigh 32%-87% more than an average human hand, making EMG hands difficult and uncomfortable to wear since the weight of such hands is applied to soft tissue instead of the skeletal system.

Conventional body and electric powered systems cannot provide actuated motion that mimics bulk skeletal muscle. This is due to linear output by motors associated with electric/intelligent systems, and linear output provided by body-powered systems that use rigid cables to transfer force and motion.

This is in contrast to bulk skeletal muscle, which generates a non-linear output under contraction/active movements and passive movements.

To overcome the issues associated with conventional actuators, academic research has developed many different types of actuators that include pneumatic or soft robotic actuators, shape memory alloys, large thermal expansion materials, combination mechanical and tissue engineered systems, thin films, and nanofibers.

Pneumatic or soft robotic actuators use compressed air or fluid to transfer into specific chambers within an actuation system, where the chambers are independent of one another. This allows the system to fill specific chambers with fluid and creates a structure that deforms to grasp and/or move objects. The downside is that these systems are complex, require a source of compressed fluid, and can be heavy relative to their size.

Shape memory alloys (SMA) and thermal expansion materials can generate high force per weight characteristics with heat by changing microstructure orientation/phase or by reversible, directional thermal expansion. A shape memory alloy is a metal alloy that “remembers” its original shape and that, when deformed, returns to its pre-deformed state when actuated (e.g., by application of electric current, heat, magnetic field, etc.).

Both SMAs and thermal expansion materials require high temperatures (e.g., up to 120° C.) for actuation/displacement, wherein such temperatures can hinder actuation response time. Such materials also exhibit low strain recovery, wherein SMAs can typically achieve a maximum strain recovery of only eight percent. Additionally, thermal expansion materials require a load to be applied to hold such materials in a deformed position so such materials can recover a shape when heated.

Additionally, mechanical/tissue engineered, thin films, nanofibers, or shape deposition manufactured actuators have been used to create actuators, but require one or more of living skeletal muscle cells, complex nanowire/fiber manufacturing and structure, or embedded electronic components. Actuators with living cells or nanofibers can generate high or physiological comparable strain rates, but have living cells that need nutrients and require complex manufacturing. Moreover, current shape deposition manufactured actuators require the use of embedded electronics during the printing process to create an actuator, but these actuators still provide a linear output response.

In consequence of the foregoing considerations, the art continues to seek improved actuators and prosthetic systems incorporating same.

### BRIEF DESCRIPTION OF DRAWINGS

FIG. 1 is a drawing depicting the layout of a fused filament fabrication (FFF) shape memory polymer (SMP) actuator having a medially arranged non-linear segment arranged in a zig-zag pattern between linear end segments that are collinear with one another.

FIG. 2A is a photograph of a FFF SMP actuator having at least one non-linear segment arranged in a zig-zag pattern between linear end segments.

FIGS. 2B-2D are 10× magnified view photographs of first, second, and third portions of the FFF SMP actuator of FIG. 2A.

FIG. 3 is a plot of tensile stress (GPa) versus strain for five samples of an extruded SMP filament.

FIG. 4 is a plot of force (Newtons) versus displacement (mm) for tensile testing of multiple samples of a FFF SMP actuator.

FIG. 5A is a chemical diagram showing the key bonds of poly-lactic acid (PLA).

FIG. 5B is a Fourier Transform Infrared (FTIR) spectroscopy plot for PLA useable in an extruded SMP filament.

FIG. 6A is a chemical diagram showing the key bonds of thermoplastic polyurethane (TPU).

FIG. 6B is a FTIR spectroscopy plot for TPU useable in an extruded SMP filament.

FIG. 7 is a plot of absorbance versus wavenumber (cm⁻¹) for SMP filament material including a blend of PLA and TPU.

FIG. 8 is a plot of absorbance versus wavenumber (cm⁻¹) for a FFF SMP actuator produced using the composite SMP filament (including a blend of PLA and TPU) characterized in FIG. 7.

FIG. 9 is a differential scanning calorimetry (DSC) curve of a SMP filament and FFF SMP actuator, for a DSC test involving heating from 0° C. to 300° C. and then cooling to 22° C. at a rate of 10° C./min.

FIG. 10 is a 45× magnification scanning electron microscopy (SEM) image taken of an oblique cross-section of an extruded SMP filament prior to FFF.

FIGS. 11A-11D are increased magnification (1,500×, 5,000×, 8,000×, and 20,000× magnification, respectively) SEM images taken of a first portion of the oblique cross-section of the extruded SMP filament of FIG. 10.

FIGS. 11E-11H are increased magnification (1,500×, 5,000×, 10,00033 , and 20,000× magnification, respectively) SEM images of a second portion of the oblique cross-section of the extruded SMP filament of FIG. 10.

FIG. 12A is a 50× magnification SEM image of an oblique cross-section taken proximate to an end segment of a FFF SMP actuator.

FIGS. 12B and 12C are 100× magnification SEM images of first and second portions of the oblique cross-section of FIG. 12A.

FIG. 13A is a 50× magnification SEM image of a straight cross-section taken proximate to an end segment of a FFF SMP actuator.

FIGS. 13B and 13C are 100× magnification SEM images of first and second portions of the straight cross-section of FIG. 13A.

FIG. 14A is a 50× magnification SEM image of an oblique cross-section taken proximate to a middle segment of a FFF SMP actuator.

FIGS. 14B and 14C are 100× magnification SEM images of first and second portions of the oblique cross-section of FIG. 14A.

FIG. 15A is a 50× magnification SEM image of a straight cross-section taken proximate to a middle segment of a FFF SMP actuator.

FIGS. 15B and 15C are 100× magnification SEM images of first and second portions of the straight cross-section of FIG. 15A.

FIG. 16A is a photograph showing four FFF SMP actuators each in a baseline condition prior to undergoing any strain cycles, with a portion of a metric rule for size reference.

FIGS. 16B-16F are photographs showing the four FFF SMP actuators of FIG. 16A after undergoing first through fifth strain cycles, respectively, during which the uppermost actuator was strained at 100%, the second actuator was strained at 80%, the third actuator was strained at 60%, and the bottom actuator was not subject to any strain.

FIG. 17 is a bar chart showing percent strain recovery versus recovery cycle number for FFF SMP actuators subjected to five cycles of 100% strain, with a superimposed line representing an average of the tested samples for each cycle.

FIG. 18 is a bar chart showing percent strain recovery versus recovery cycle number for FFF SMP actuators subjected to five cycles of 80% strain, with a superimposed line representing an average of the tested samples for each cycle.

FIG. 19 is a bar chart showing percent strain recovery versus recovery cycle number for FFF SMP actuators subjected to five cycles of 60% strain, with a superimposed line representing an average of the tested samples for each cycle.

FIG. 20 is a bar chart showing percent strain recovery versus recovery cycle number for FFF SMP actuators subjected to five cycles of 40% strain, with a superimposed line representing an average of the tested samples for each cycle.

FIG. 21 is a bar chart showing percent strain recovery versus recovery cycle number for FFF SMP actuators subjected to five cycles of 20% strain, with a superimposed line representing an average of the tested samples for each cycle.

FIG. 22 is a plot of average percent recovery versus strain percentage for strain percentages of 100%, 80%, 60%, 40%, and 20%, with horizontal bars representing the minimum and maximum strain values for cycles 1-5.

FIGS. 23A-23E are time stepped thermal images (at 0 sec., 0.33 sec., 0.66 sec., 0.77 sec., and 1.10 sec., respectively) showing the onset of shape memory effect of an SMP actuator with a layer height of 1.5 mm upon exposure to 70° C. deionized (DI) water, with FIGS. 23D and 23E including a superimposed black circle showing the onset of shape recovery of the SMP actuator.

FIG. 24 is a line chart plotting force (N) versus temperature (° C.) for SMP actuators subjected to 100% strain for five cycles and contacted with heated water.

FIG. 25 is a line chart plotting force (N) versus temperature (° C.) for SMP actuators subjected to 80% strain for five cycles and contacted with heated water.

FIG. 26 is a line chart plotting force (N) versus temperature (° C.) for SMP actuators subjected to 60% strain for five cycles and contacted with heated water.

FIG. 27 is a line chart plotting force (N) versus temperature (° C.) for contact of a SMP actuator with a ceramic heater and showing each strain for five cycles with the SMP actuator subjected to 60% strain.

FIG. 28 is a line chart plotting force (N) versus temperature (° C.) for contact of SMP actuators with heated water and showing each strain for five cycles with the SMP actuators subjected to 40% strain and 20% strain, respectively.

FIG. 29 is a line chart plotting force (N) versus temperature (° C.) for contact of SMP actuators with heated water and showing strain only for the first and fifth cycles, with the SMP actuator subjected to 100% strain.

FIG. 30 is a line chart plotting force (N) versus temperature (° C.) for contact of SMP actuators with heated water and showing strain only for the first and fifth cycles, with the SMP actuator subjected to 80% strain.

FIG. 31 is a line chart plotting force (N) versus temperature (° C.) for contact of SMP actuators with heated water and showing strain only for the first and fifth cycles, with the SMP actuator subjected to 60% strain.

FIG. 32 is a line chart plotting force (N) versus temperature (degrees Celsius) for contact of a SMP actuator with a ceramic heater and showing strain only for cycles 1 and 5, with the SMP actuator subjected to 60% strain.

FIG. 33 is a plot of average stress (MPa) versus strain percentage (%) for five cycles including contact of SMP actuators with heated water at each strain percentage of 100%, 80%, 60%, 40%, and 20%, and for five cycles including contact of a SMP actuator with a ceramic heater at 60% strain.

FIG. 34 is a plot of average stress (MPa) versus stretch cycle number for strain percentage (%) for five cycles including contact of SMP actuators with heated water at each strain percentage of 100%, 80%, 60%, 40%, and 20%, and for five cycles including contact of a SMP actuator with a ceramic heater at 60% strain.

FIG. 35 is a plot of average slope (MPa/degree Celsius) versus strain percentage (%) for five cycles including contact of SMP actuators with heated water at each strain percentage of 100%, 80%, 60%, 40%, and 20%, and for five cycles including contact of a SMP actuator with a ceramic heater at 60% strain.

FIG. 36 is a plot of slope (MPa/degree Celsius) versus stretch cycle number for strain percentage (%) for five cycles including contact of SMP actuators with heated water at each strain percentage of 100%, 80%, 60%, 40%, and 20%, and for five cycles including contact of a SMP actuator with a ceramic heater at 60% strain.

FIG. 37 is a schematic illustration of a prosthetic device including a movable joint configured to permit pivotal movement between first and second structural members, with a first group of thermally responsive shape memory actuators coupled between first and third anchor elements to promote pivotal movement in a first direction, and with a first group of thermally responsive shape memory actuators coupled between second and fourth anchor elements to promote pivotal movement in a second direction that differs from the first direction.

### SUMMARY

Disclosed herein are novel thermally responsive shape memory polymer (SMP) actuators, and prosthetic devices incorporating multiple thermally responsive SMP actuators, as well as methods for their fabrication and use. SMP actuators may be used to provide movement and force to joints within a prosthetic device.

In one aspect of the disclosure, a thermally responsive shape memory actuator comprises a body including a first end, a second end, and at least one non-linear segment disposed between the first end and the second end. The body comprises a plurality of fused shape memory polymer elements comprising a plurality of dots, rods, or layers.

In certain embodiments, the non-linear segment comprises a substantially flat zig-zag shape.

In certain embodiments, the body comprises a first substantially straight segment proximate to the first end and a second substantially straight segment proximate to the second end, with the at least one non-linear segment being arranged between the first substantially straight segment and the second substantially straight segment.

In certain embodiments, the plurality of fused shape memory polymer elements comprises a linear aliphatic thermoplastic polyester and at least one other polymer. In certain embodiments, the at least one other polymer comprises a melting temperature that exceeds a melting temperature of the linear aliphatic thermoplastic polyester.

In certain embodiments, the linear aliphatic thermoplastic polyester comprises at least one of a poly(L-lactide)-based polymer or a poly(ε-caprolactone)-based polymer. In certain embodiments, the plurality of fused shape memory polymer elements comprises poly(L-lactide) and thermoplastic polyurethane.

In certain embodiments, the plurality of fused shape memory polymer elements comprises poly(ε-caprolactone) and at least one other polymer. In certain embodiments, the at least one other polymer comprises polyhedral oligosilsesquioxane.

In certain embodiments, the body is pre-strained by heating and elongation in a range of 140% to 170% of an initial length of the body.

In another aspect of the disclosure, a prosthetic device comprises a plurality of thermally responsive shape memory actuators, a movable joint connected between first and second structural members, and multiple anchors associated with the structural members. Each thermally responsive shape memory actuator comprises a body including a non-linear segment disposed between two body ends, and the body comprises a shape memory polymer material. The movable joint is configured to permit pivotal movement between the first structural member and the second structural member. A first anchor element and a second anchor element are associated with the first structural member, and a third anchor element and a fourth anchor element are associated with the second structural member. A first group of thermally responsive shape memory actuators is coupled between the first anchor element and the third anchor element, and is configured to promote pivotal movement between the first structural member and the second structural member in a first direction. Additionally, a second group of thermally responsive shape memory actuators is coupled between the second anchor element and the fourth anchor element, and is configured to promote pivotal movement between the first structural member and the second structural member in a second direction that differs from the first direction.

In certain embodiments, the body of each thermally responsive shape memory actuator of the plurality of thermally responsive shape memory actuators is produced by a method including at least one additive manufacturing step. In certain embodiments, the body of each thermally responsive shape memory actuator of the plurality of thermally responsive shape memory actuators comprises a plurality of fused shape memory polymer elements comprising a plurality of dots, rods, or layers.

In certain embodiments, the body of each thermally responsive shape memory actuator of the plurality of thermally responsive shape memory actuators is produced by a method including at least one molding step.

In certain embodiments, the body of each thermally responsive shape memory actuator of the plurality of thermally responsive shape memory actuators is produced by a method including at least one subtractive manufacturing step.

In certain embodiments, for each thermally responsive shape memory actuator of the plurality of thermally responsive shape memory actuators, the body includes a first end, a second end, and at least one non-linear segment disposed between the first end and the second end. In certain embodiments, the non-linear segment comprises a substantially flat zig-zag shape.

In certain embodiments, for each thermally responsive shape memory actuator of the plurality of thermally responsive shape memory actuators, the body comprises a first substantially straight segment proximate to the first end and a second substantially straight segment proximate to the second end, with the at least one non-linear segment being arranged between the first substantially straight segment and the second substantially straight segment.

In certain embodiments, for each thermally responsive shape memory actuator of the plurality of thermally responsive shape memory actuators, the shape memory polymer material of the body comprises a linear aliphatic thermoplastic polyester and at least one other polymer. In certain embodiments, the at least one other polymer comprises a melting temperature that exceeds a melting temperature of the linear aliphatic thermoplastic polyester.

In certain embodiments, the linear aliphatic thermoplastic polyester comprises at least one of a poly(L-lactide)-based polymer or a poly(ε-caprolactone)-based polymer.

In certain embodiments, the shape memory polymer material of the body comprises poly(L-lactide) and thermoplastic polyurethane.

In certain embodiments, the shape memory polymer material of the body comprises poly(ε-caprolactone) and at least one other polymer. In certain embodiments, the at least one other polymer comprises polyhedral oligosilsesquioxane.

In certain embodiments, the body of each thermally responsive shape memory actuator of the plurality of thermally responsive shape memory actuators is pre-strained by heating and elongation in a range of 140% to 170% of an initial length of the body.

In another aspect of the disclosure, a method of fabricating a thermally responsive shape memory actuator, the method comprising: forming a body by additive manufacturing, the body comprising a shape memory polymer material, a first end, a second end, and at least one non-linear segment disposed between the first end and the second end; following formation of the body, heating the body into a glass transition temperature range of the shape memory polymer material; applying tension to the body while the body is at an elevated temperature due to said heating of the body, wherein the tension is sufficient to elongate the body by at least partial straightening of the at least one non-linear segment; and cooling the body.

In certain embodiments, the additive manufacturing comprises fused filament fabrication.

In certain embodiments, the additive manufacturing comprises stereolithography, selective laser sintering, or selective laser melting.

In certain embodiments, the body comprises a plurality of fused shape memory polymer elements comprising a plurality of dots, rods, or layers.

In certain embodiments, the tension is sufficient to elongate the body by 40% to 70% relative to an initial length of the body, to yield an aggregate (elongated) length of 140% to 170% of the initial length.

In certain embodiments, the body comprises a first substantially straight segment proximate to the first end and a second substantially straight segment proximate to the second end, with the at least one non-linear segment being arranged between the first substantially straight segment and the second substantially straight segment.

In certain embodiments, the elevated temperature is within the glass transition temperature range of the shape memory polymer material. In certain embodiments, the elevated temperature is within about 10% of the glass transition temperature range of the shape memory polymer when the glass transition temperature range is expressed in Kelvin.

In another aspect, any one or more aspects or features described herein may be combined with any one or more other aspects or features for additional advantage.

### DETAILED DESCRIPTION

Disclosed herein are novel thermally responsive shape memory actuators, and prosthetic devices incorporating multiple thermally responsive shape memory actuators, as well as methods for their fabrication and use. In certain embodiments, a thermally responsive shape memory actuator body includes at least one non-linear segment disposed between a first end and a second end, wherein the body includes a plurality of fused shape memory polymer (SMP) elements comprising a plurality of dots, rods, or layers. A method of fabricating a thermally responsive shape memory actuator includes forming, by additive manufacturing, a body of SMP material including at least one non-linear segment disposed between a first end and a second end, followed by heating the body into a glass transition temperature range of the SMP material, then applying tension to the body while the body is at an elevated temperature due to said heating of the body, wherein the tension is sufficient to elongate the body by at least partial straightening of the at least one non-linear segment, and then cooling the body. Further disclosed herein is a prosthetic device including a first group of thermally responsive SMP actuators configured to promote pivotal movement between first and second structural members in a first direction, and including a second group of thermally responsive SMP actuators configured to promote pivotal movement between the first and second structural members in a second direction that differs from the first direction.

SMP actuators disclosed herein may be used, for example, as biomimicking skeletal muscle actuators for upper-limb prosthetics. Actuators are used to provide movement and force to joints within a prosthetic device. For upper-limb prosthetics, this would include digit/wrist manipulation, grip force, and rotation at the elbow. SMP actuators disclosed herein desirably exhibit non-linear response properties, peak force and strain comparable to mammalian skeletal muscle, a rapid response time (e.g., 0.77 sec), low operating temperature of 70° C., and a low mass (e.g., 74.0 mgrams), low volume (e.g., 46.74 mm³), and low material costs (e.g., less than 1 cent, or $0.0098 per SMP actuator).

SMP actuators according to certain embodiments may be produced by at least one additive manufacturing step, such as three-dimensional printing. One example of an additive manufacturing process is fused filament fabrication or “FFF” (a/k/a fused deposition modeling). Further examples of additive manufacturing processes include stereolithography, selective laser sintering, and selective laser melting. Various additive manufacturing steps are suitable to yield a plurality of fused shape memory polymer elements that may comprise a plurality of dots, rods, or layers. In other embodiments, at least one subtractive manufacturing step (e.g., machining, laser cutting, laser ablation, etc.) may be used to remove material from a larger mass to result in a product of a desired size and shape.

SMP actuators according to at least certain embodiments herein are designed to overcome the issues of high operating temperatures, low contractile strain, complexity, high weight and cost, and linear output of conventional actuators. In certain embodiments, a SMP actuator exhibits non-linear contractile and passive forces, contractile forces comparable to mammalian skeletal muscle, reaction time under one second, low operating temperature, high contractile strain, low cost, low mass, and low volume.

Shape memory polymers are able to recover a predetermined or memorized shape (shape memory effect) by shaping the SMP at room temperature or in its glass transition temperature (Tg) range. Manipulating the SMP at its Tg allows the SMP to be easily shaped and/or deformed, since the SMP has transitioned from a hard/glassy state (characteristic of low temperature) to a soft/rubbery state in which the polymer chains can be rearranged. The SMPK is then cooled (shape fixity) in its deformed shape until it returns to a glassy state. Thereafter, when the polymer is heated to its Tg, it recovers its original shape/polymer chain configuration.

In certain embodiments, a SMP material comprises a linear aliphatic thermoplastic polyester and at least one other polymer. In certain embodiments, the at least one other polymer comprises a melting temperature that exceeds a melting temperature of the linear aliphatic thermoplastic polyester. In certain embodiments, the linear aliphatic thermoplastic polyester comprises at least one of a poly(L-lactide)-based polymer or a poly(ε-caprolactone)-based polymer. In certain embodiments, the SMP material comprises poly(L-lactide) and thermoplastic polyurethane. In certain embodiments, the SMP material comprises poly(ε-caprolactone) and at least one other polymer; optionally, the at least one other polymer comprises polyhedral oligosilsesquioxane.

In certain embodiments, a SMP actuator may comprise a melt-blended mixture of poly-lactic acid (PLA) and thermoplastic polyurethane (TPU) in a 7:3 ratio respectively. Although PLA and TPU both demonstrate shape memory effects, blending them together introduces additional elasticity from the TPU and shape fixity at room temperature from the PLA. The composite SMP material may then be manufactured in a 2D spring shape susceptible to use of radiating heat to generate actuation. Using a SMP material allows the SMP actuator to memorize a predetermined shape and recover that shape with the application of heat when the SMP is deformed and/or displaced.

An exemplary SMP composite material was created using PLA and TPU filament purchased from SainSmart. The filament was cut to pellets that were aerated in a funnel with compressed air, with a film of 99.9% isopropyl-alcohol, and grounding wire to minimize static charge buildup. Aeration was performed by applying 100.0 psi compressed air with three second bursts for 60 seconds with 28.5 grams of composite mixture until a homogeneous mixer was obtained. The mixture was then extruded into a filament with the use of a single screw extruded with a 1.5 mm nozzle diameter. (Had a dual screw extruder been used, the above-described mixing step may have been avoided.) The extruder had a nozzle temperature of 180.0° C. with a screw rotation speed of 0.037 revolutions/sec. Aerating with a lower or higher air pressure would not allow for thorough pellet mixing. The lower pressure resulted in an increase in mixing time, which allowed for the isopropyl-alcohol film to wear off and then static charge would begin to build. Such a condition resulted in clustering and separation of pellets from the mixture. The higher pressure also resulted in a loss of film due to the increase in pellet friction during aeration.

SMP actuators were designed using SOLIDWORKS 3D CAD software and samples were produced by fused filament fabrication (FFF) using a MakerGear M2 printing apparatus aided with Simplify3D software. The SMP actuators had a cross-section of 1.5 mm×0.4 mm, overall length and width of 50.0 mm and 12.38 mm, and were printed with a 0.35 mm diameter nozzle at 240.0° C. with the bed at 70.0° C. Software was coded to print with an: extrusion multiple of 0.97, extrusion width of 0.39, layer height of 0.3 mm, and a printing speed of 600.0 mm/min.

As used herein, the term “substantially straight” as used refers to a segment that is either straight between two endpoints thereof, or a segment having a slightly curved or non-linear portion in which all intermediate points thereof fall within range departing no more than ±7.5 degrees from a straight line drawn between two endpoints of the segment.

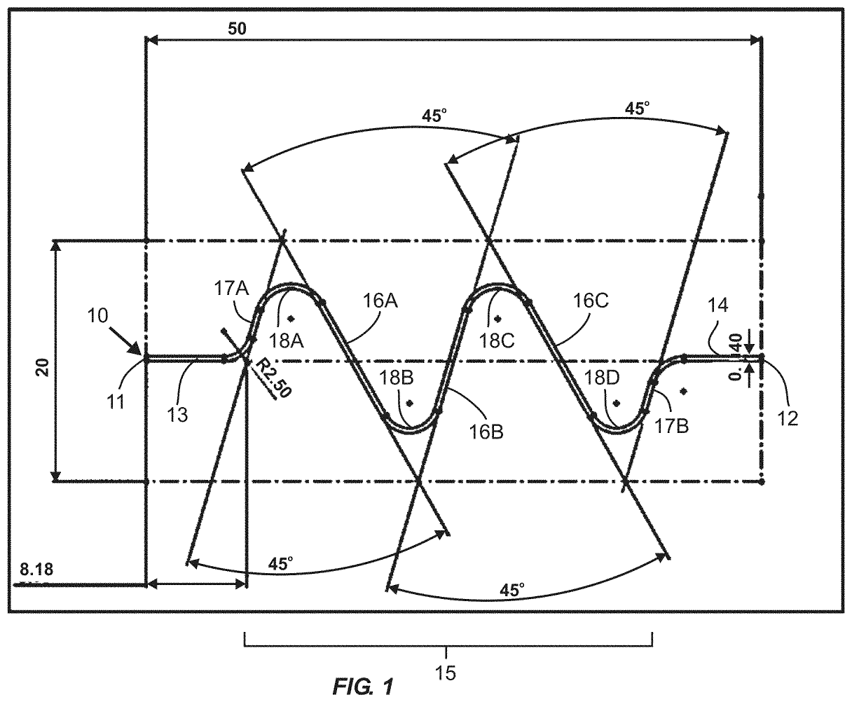

FIG. 1 is a drawing depicting the layout of a FFF shape memory polymer (SMP) actuator 10 having at least one non-linear segment 15 arranged in a zig-zag pattern between linear end segments 13, 14 that are collinear with one another. As shown, the SMP actuator has a length of 50 mm from a first end 11 to a second end 12; each linear end segment 13, 14 has a length of about 8 mm; and the SMP actuator 10 has a thickness of about 0.4 mm. The at least one non-linear segment 15 includes three full-length portions 16A-16C and two reduced length portions 17A, 17B that are each oriented 45 degrees relative to one another and are connected by four turns 18A-18D.

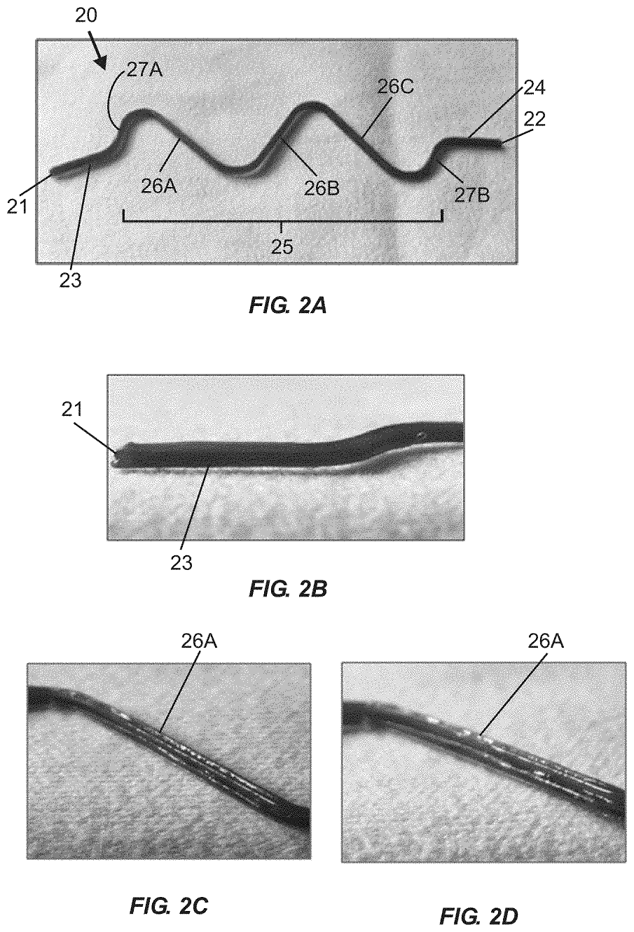

FIG. 2A is a photograph of a FFF SMP actuator 20 having at least one non-linear segment 25 arranged in a zig-zag pattern between first and second linear end segments 23, 24 that terminate at first and second ends 21, 22, respectively. The at least one non-linear segment 25 includes three full-length portions 26A-26C arranged between two reduced length portions 27A, 27B. The non-linear segment 25 of the FFF SMP actuator 20 enables the FFF SMP actuator 20 to be elongated beyond its initial length to a certain extent without incurring high tensile stress that would lead to thinning and breakage of the actuator material. Due to irregularities during fabrication and/or thermal cycling, the linear end segments 23, 24 are not collinear with one another in FIG. 2A.

FIGS. 2B and 2C are 10× magnified view photographs of portions of the FFF SMP actuator 20 of FIG. 2A. FIG. 2B shows the first end segment 23 and first end 21 of the FFF SMP actuator 20, while FIG. 2B shows a first full-length portion 26A of the at least one non-linear segment 25. FIG. 2D is a 20× magnified view photograph of the first full-length portion 26A of the at least one non-linear segment 25. In FIGS. 2B-2D, lines extending in a longitudinal direction are visible, corresponding to boundaries between deposited SMP layers of the FFF SMP actuator.

Physical and chemical material properties of FFF SMP actuators were acquired using of Fourier-transform infrared-spectroscopy (FTIR), differential scanning calorimetry (DSC), scanning electron microscope (SEM), and tensile testing. SEM images were taken of sections at the ends and middle of SMP actuator samples. Sections were made by freezing the samples at −10° C. and the cutting with a razor blade at 90° and 45° (with the 45° cuts resulting in oblique cross-sections). Tensile testing used an MTS Model Sintech 1/S tensile machine (maximum load cell capability of 900 lbs). Actuator force data was collected with an iWorx data logger, a 250 gram force transducer, and a 100° C. K-type thermocouple transducer to monitor force and temperature simultaneously.

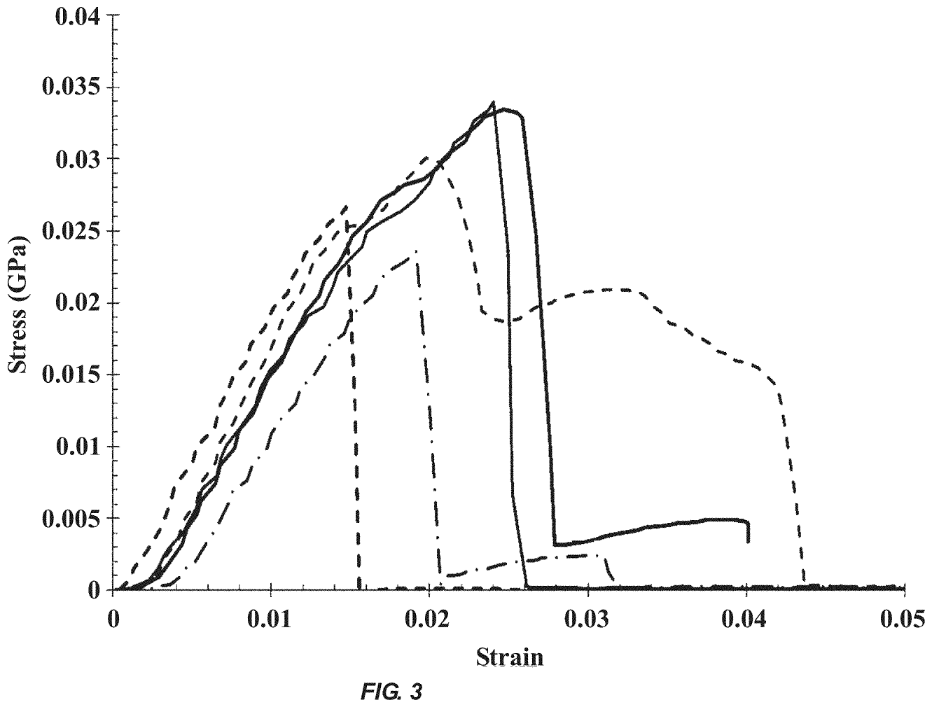

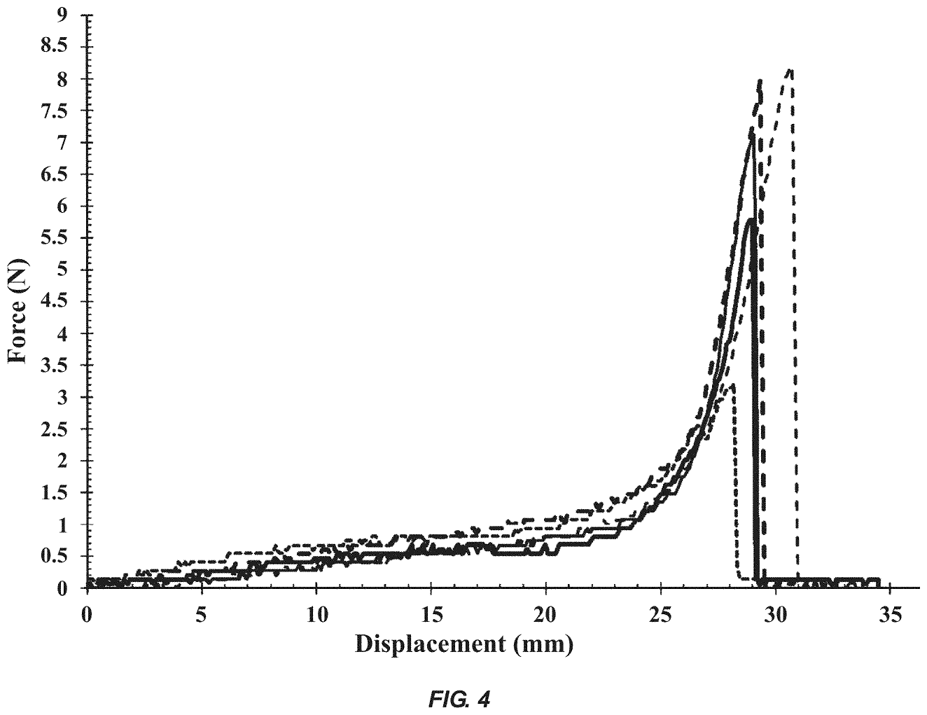

To determine mechanical properties of the composite SMP filament used to produce the FFF SMP actuators, the composite SMP filament was tensile tested to show its mechanical material property at a loading rate of 50 mm/min, following the ASTM D638 standard. Five samples were tested in tension and showed that the SMP filament is brittle, due to the 70% PLA matrix content. FIG. 3 is a plot of tensile stress versus strain of an extruded SMP filament, with a sample size of five. Testing showed a consistent linear elastic region with fluctuations in peak stress ranging from 0.0337 GPa to 0.0236 GPa, as shown in FIG. 3. These fluctuations may have been caused by clustering, where a large cluster may cause a high stress or weakening point in the sample and cause it to fail early. Fluctuations in peak force appear to be indications of TPU clusters seen in SEM imaging. FIG. 4 is a plot of force (Newtons) versus displacement (mm) for tensile testing of a FFF SMP actuator. In addition to the fluctuations in peak stress, two samples showed a slight increase in ductility with one sample having a secondary peak. The increase in ductility and the secondary peak are due to a particle or cluster of TPU material within the cross-section. Even though SEM imaging showed an evenly distributed composite, if the point of failure in the filament has a cluster of TPU, then the sample can demonstrate a secondary tensile peak. The increase in ductility is minimal in these samples and does not affect the elastic tension regions.

With continued reference to FIG. 4, as the SMP actuator is displaced it initially provides minimal resistance, and then has an exponential increase in resistance to displacement when nearing the complete straightening of the SMP actuator. This spring design allows for the SMP actuator to have non-linear properties and behave comparable to skeletal muscle in passive movement, strain/shape recovery, and peak force generation. Restated, the tensile testing results show that as a SMP actuator is displaced, there is a shallow force uptake, followed by a steep absorption of force, and then failure at 30 mm of displacement, with such progression following the non-linear passive tension that skeletal muscles demonstrate. The SMP actuator only works within the elastic region of the polymer, since going above the elastic region would cause plastic deformation in the SMP actuator.

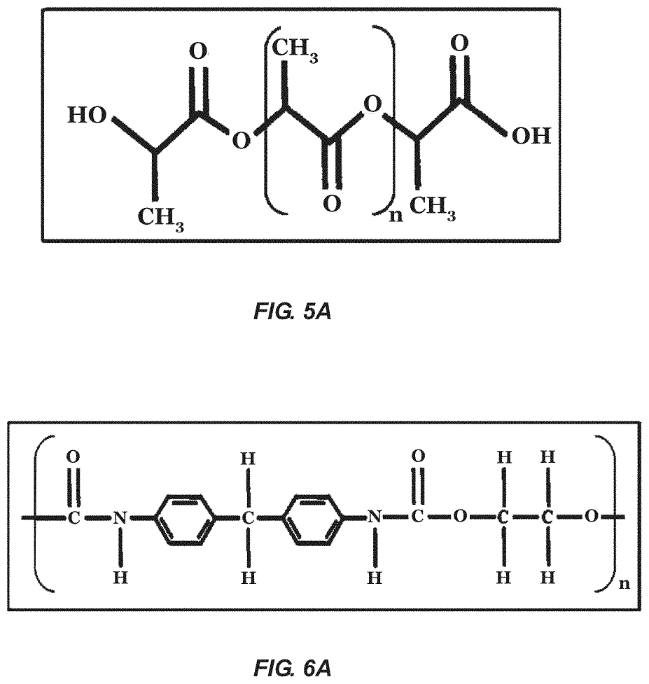

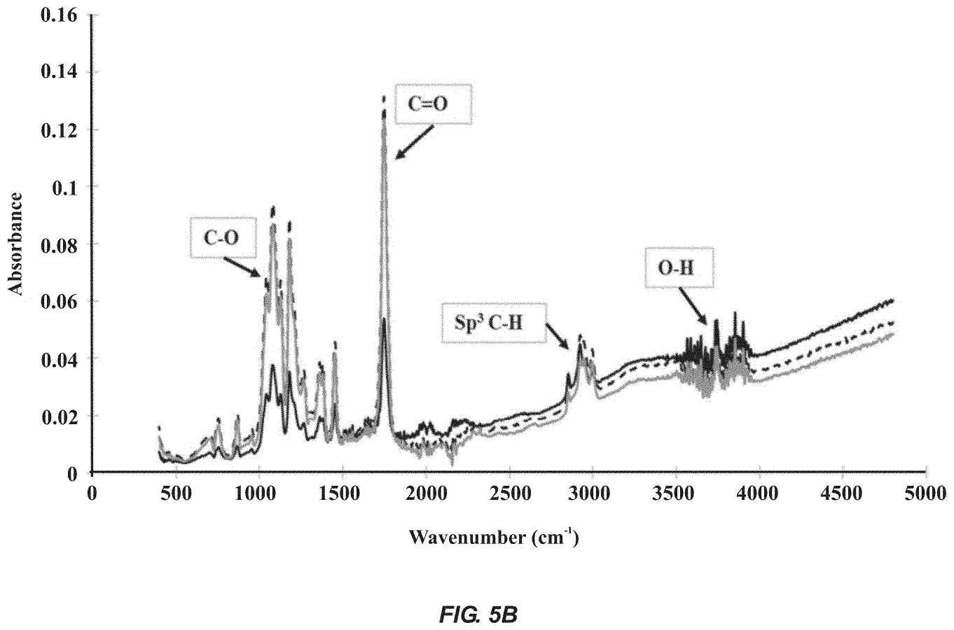

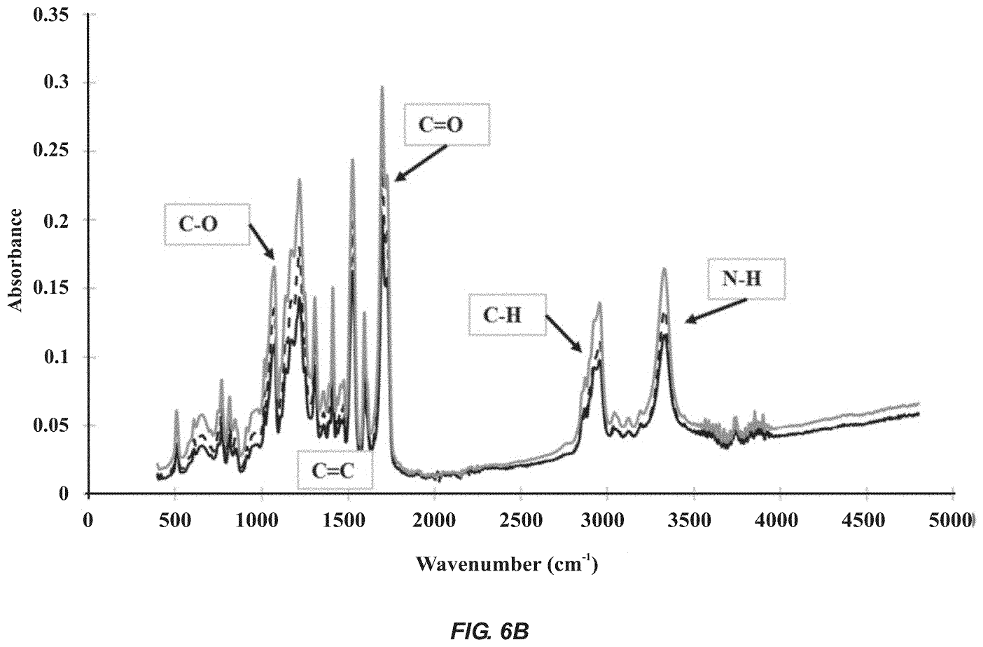

To verify material composition of the composite material used to fabricate the SMP actuators, after extrusion of the SMP filament, the material was tested with a Fourier Transform Infrared (FTIR) spectroscopy machine to identify that both PLA and TPU were present in the melt mixed blend. FIGS. 5A and 6A are chemical diagrams showing the key bonds of PLA and TPU, respectively. FTIR samples of PLA, TPU, and composite SMP filament were tested with a sample size of three with sweeps from 4800 cm⁻¹ to 400 cm⁻¹ to show that the SMP actuator still had a blended polymer matrix. FIGS. 5B and 6B are FTIR spectroscopy plots for PLA and TPU, respectively, useable in an extruded composite SMP filament. FIG. 7 is a plot of absorbance versus wavenumber (cm⁻¹) for SMP filament material including a blend of PLA and TPU.

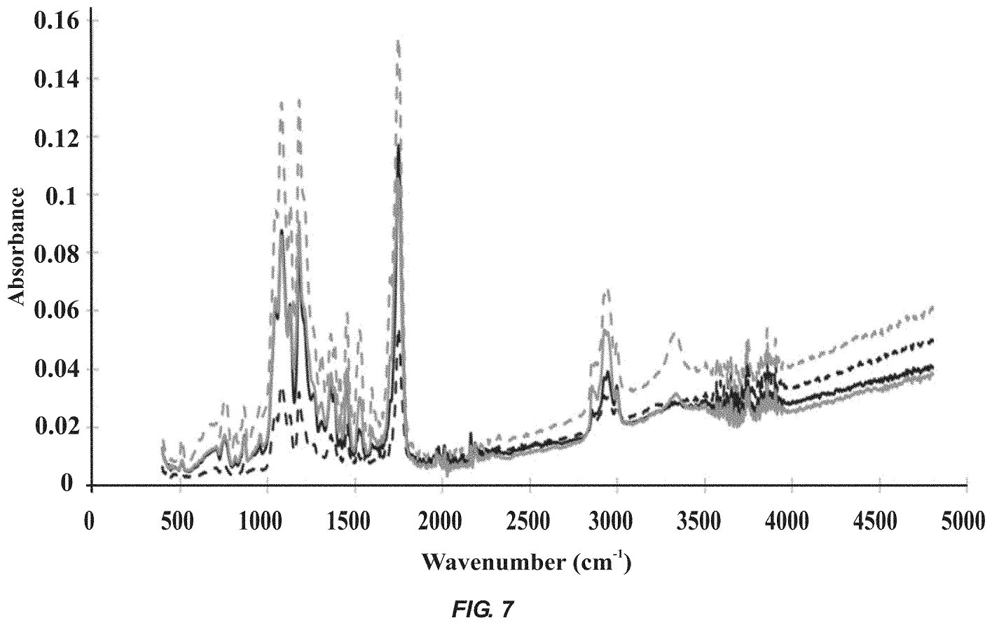

FIG. 8 is a plot of absorbance versus wavenumber (cm⁻¹) for a fused filament fabrication SMP actuator produced using the composite SMP filament characterized in FIG. 7.

Initial FTIR and tensile material analysis showed that the extruded SMP filament and FFF SMP actuators had the appropriate carbon (C), hydrogen (H), oxygen (O), nitrogen (N), Spa carbon to hydrogen, and carbon to carbon or oxygen double bonds for PLA and TPU, as shown in FIGS. 5A-8. The FTIR results show the amount of infrared-radiation (IR) absorbed versus wavenumber. This is obtained since absorbed infrared radiation causes a molecule to be excited into a higher vibrational state, where the change in energy from the at-rest state to the excited molecule state is a function of the amount of absorption at a wavenumber by a particular molecule/bond. FTIR results show that all key bonds are present in all samples and types.

To determine the quality of the FFF SMP actuators and if any thermal degradation of the SMP material resulted after production by FFF, DSC testing and SEM imaging were performed. More specifically, DSC was used to determine polymer degradation, and SEM was used to evaluate layer adhesion as well as 3D print quality of the FFF SMP actuators.

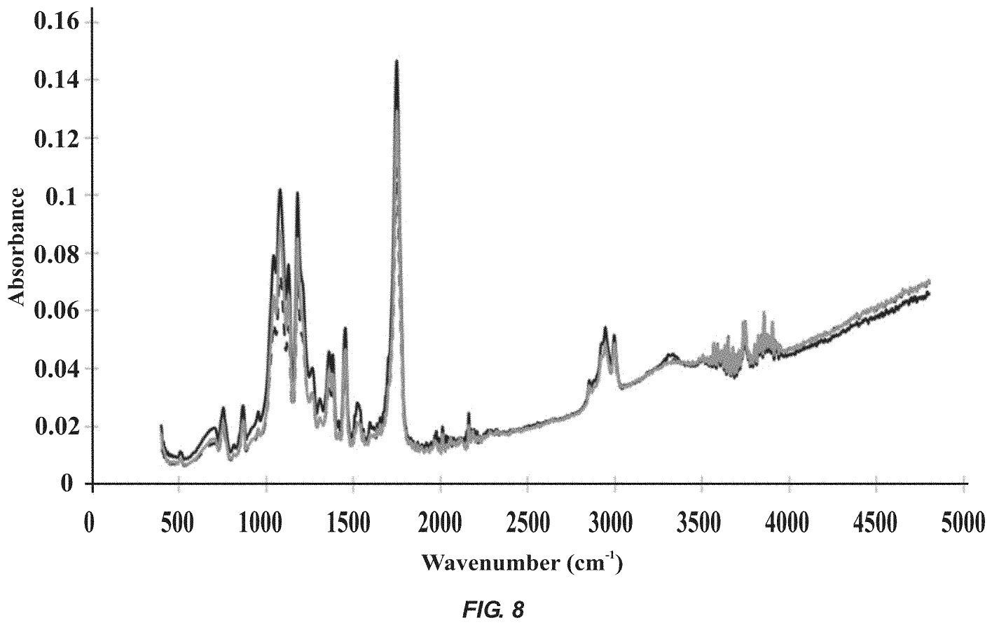

FIG. 9 is a differential scanning calorimetry (DSC) curve of a SMP filament and FFF SMP actuator. The DSC test involved heating from 0° C. to 300° C. and then cooling to 22° C. at a rate of 10°/min. The results of the DSC test, depicted in FIG. 9, show a glass transition (Tg) endothermic event first, followed by a cold crystallization exothermic event second, and then the melting temperature (Tm) endothermic event. Upon cooling from 300° C., an exothermic event is seen due to crystallization from annealing. An endothermic event represents an absorption of heat into the sample and an exothermic event represents the release of heat from the sample.

The cold crystallization event is present since the content of the SMP is 70% PLA, which is an amorphous semi-crystalline polymer. The TPU has a pseudo crystal structure in which the hard segments within the polymer are ordered and the soft segments are amorphous. Once the SMP reaches its Tg, the polymer-chains reorder themselves and increase their crystallization, inducing an exothermic event. If the polymer had high crystallinity originally, then the exothermic event would be minimal or not present.

When comparing the DSC cycles from the SMP filament to FFF actuator, it is apparent that thermal degradation is present due to the decrease in specific thermal energy (area under the curve) in each endothermic phase, along with the absence of an exothermic event during the transition into the Tg when annealing from 300° C. Restated, there is a decrease in thermal energy required to progress through each material phase when the filament is extruded again for FFF. The glass transition and melting temperatures are similar, but the decrease in heat flow/specific thermal energy demonstrates that there is less energy needed to go from one state to the next due to thermal degradation. The thermal degradation is caused by oxidation and random chain scission due to thermal cycling, which decreases the molecular weight. Furthermore, there is spike in exothermic energy during annealing of the filament that is not seen in the FFF SMP actuator.

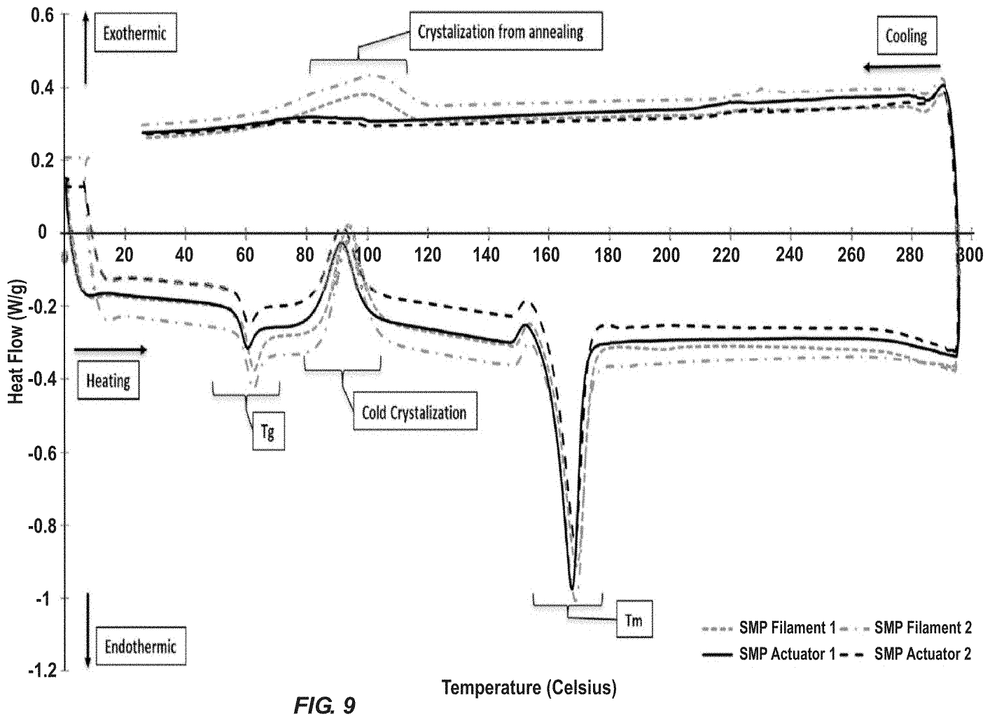

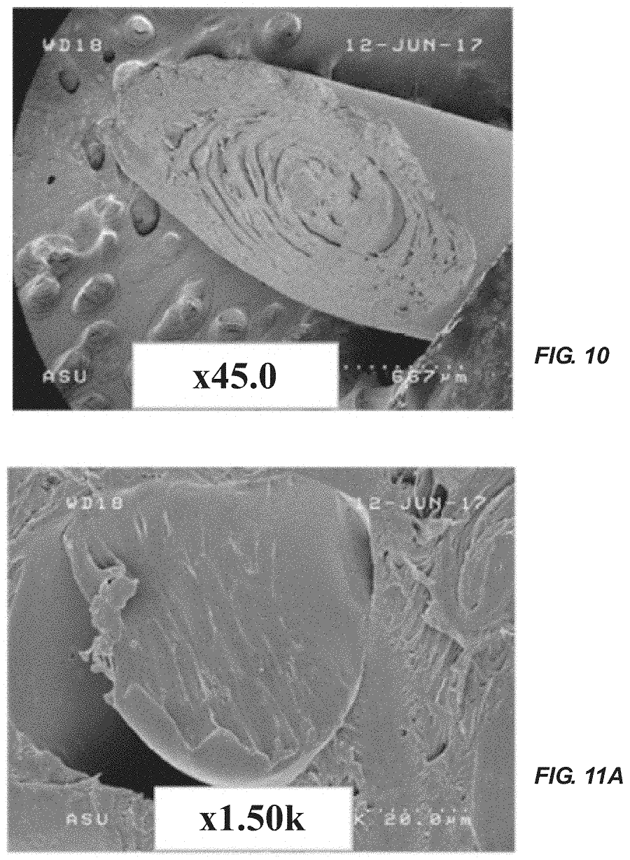

SEM imaging was useful to evaluate the distribution of TPU within the PLA matrix, since PLA makes up 70% of the blend. FIG. 10 is a 45× magnification scanning electron microscopy (SEM) image taken of an oblique cross-section of an extruded SMP filament prior to FFF. The oblique cross-sections included a cut of approximately 45 degrees through the filament. FIGS. 11A-11D are increased magnification (1,500×, 5,000×, 8,000×, and 20,000× magnification, respectively) SEM images taken of a first portion of the oblique cross-section of the extruded SMP filament of FIG. 10. FIGS. 11E-11H are increased magnification (1,500×, 5,000×, 10,000×, and 20,000× magnification, respectively) SEM images of a second portion of the oblique cross-section of the extruded SMP filament of FIG. 10.

In FIGS. 10-11H, the samples were coated with a gold/palladium film to remove charge from the surface during imaging. The dark regions or areas correspond to PLA, where the lighter or white highlights or regions correspond to TPU. There is even distribution of the TPU within the matrix, with possible regions of TPU clustering. The imaging shows possible clustering within the matrix since the TPU particles are spherical or isolated instead of elongated and distributed. The clustering effect could have been caused by using a single-screw extruder with a premixed pellet blend, where use of a twin-screw extruder with simultaneous blending could possibly resolve the particle isolation issue.

This clustering effect appears to have influenced the tensile peak results of the SMP filament.

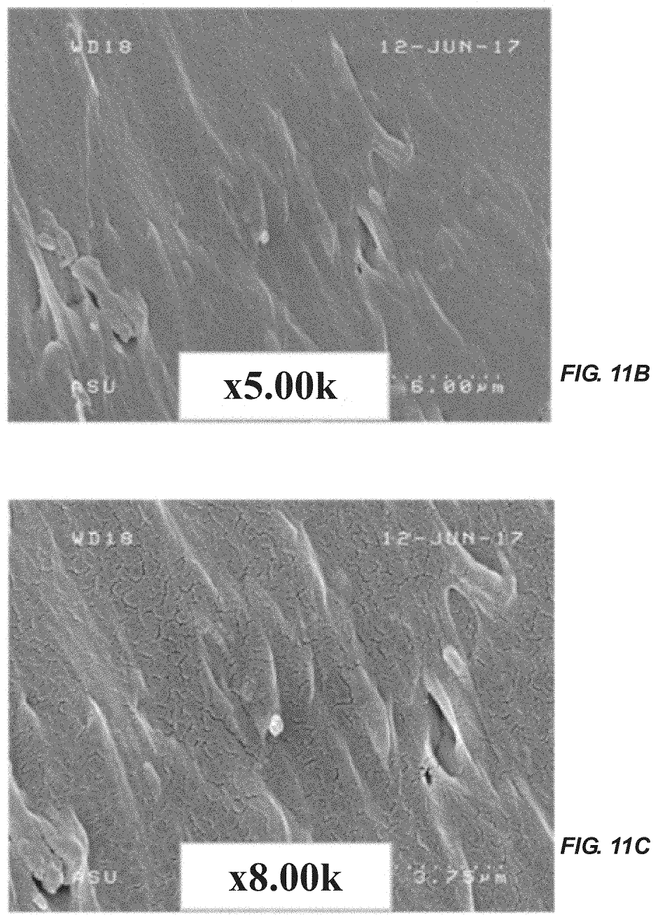

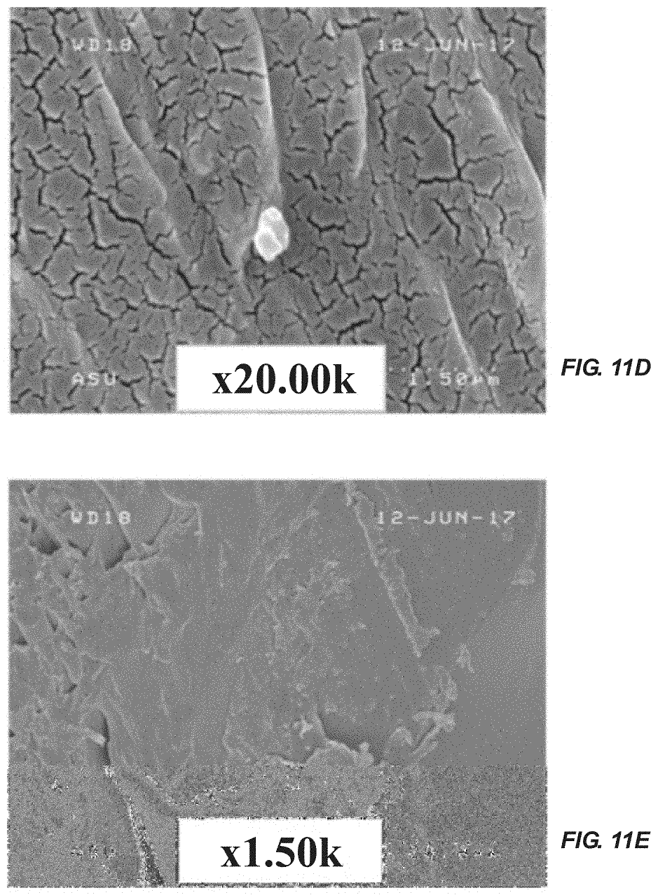

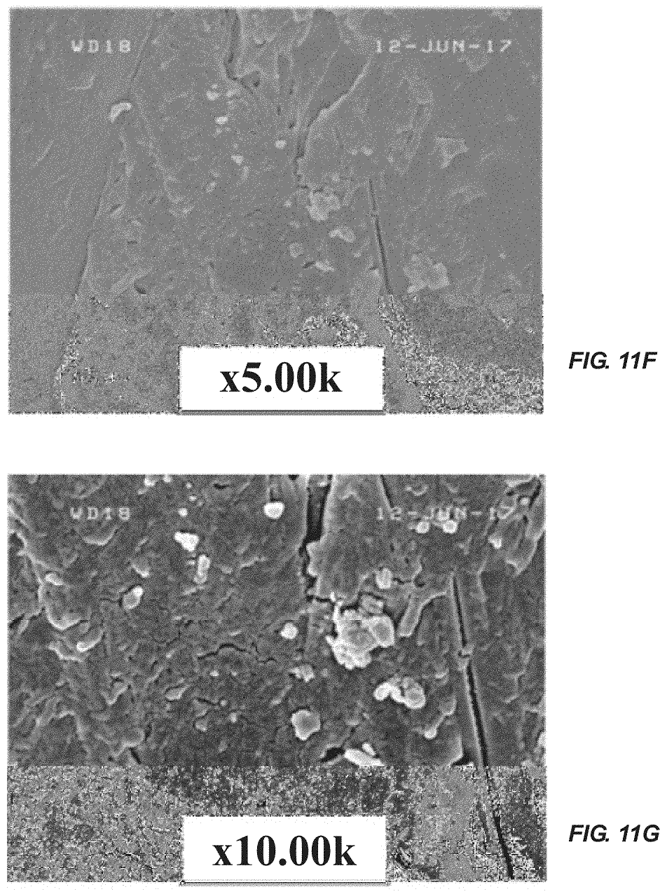

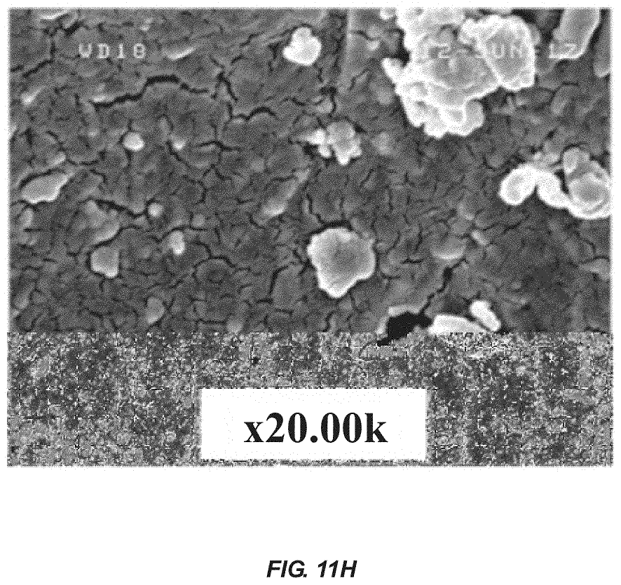

FIGS. 12A-15C embody SEM images of various cross-sections of a FFF SMP actuator. FIG. 12A is a 50× magnification SEM image of an oblique cross-section taken proximate to an end segment of a FFF SMP actuator. FIGS. 12B and 12C are 100× magnification SEM images of first and second portions of the oblique cross-section of FIG. 12A. FIG. 13A is a 50× magnification SEM image of a straight cross-section taken proximate to an end segment of a FFF SMP actuator. FIGS. 13B and 13C are 100× magnification SEM images of first and second portions of the straight cross-section of FIG. 13A. FIG. 14A is a 50× magnification SEM image of an oblique cross-section taken proximate to a middle segment of a FFF SMP actuator. FIGS. 14B and 14C are 100× magnification SEM images of first and second portions of the oblique cross-section of FIG. 14A. FIG. 15A is a 50× magnification SEM image of a straight cross-section taken proximate to a middle segment of a FFF SMP actuator. FIGS. 15B and 15C are 100× magnification SEM images of first and second portions of the straight cross-section of FIG. 15A.

The foregoing FIGS. 12A-15C show that the FFF SMP actuator has an inconsistent cross-sectional area (CSA), and exhibits inconsistency in density and layer adhesion. Images were taken at the ends and center of the actuator, and revealed that the layer adhesion is inconsistent throughout the actuator and causes layer separation. Layer separation is caused by the cooling of the layer beneath prior to the next layer being extruded thereon, and are displayed as parting lines or gaps in the image. Additionally, the cross-section of the SMP actuator is composed of peaks and valleys instead of straight lines, which causes a variation of the CSA throughout the sample.

To determine the shape recovery (SR) characteristics of FFF SMP actuator samples, samples were stretched to varying pre-determined strain values. In particular, a water bath of 70.0° C. was used to strain each sample to pre-determined strain values of 100%, 80%, 60%, 40%, and 20%, each representing a percentage elongation relative to an initial length. The strain values used show the range of capable displacement values for the FFF SMP actuator, since straining above 100% causes fracture in the SMP actuator during stretching. The strain values of 100%, 80%, 60%, 40%, and 20% represent displacement values of 50 mm, 40 mm, 30 mm, 20 mm, and 10 mm respectfully. The water bath provided a constant temperature reservoir along with fast shape fixing and recovery setting times.

The range of predetermined strain values was to demonstrate the range of recovery force with varying strain input, while 70° C. was used since the glass transition temperature of PLA is 60-65° C. Additionally, each sample was cyclically fatigued by stretching and recovering its shape at 70.0° C. for five cycles, with different samples being subjected to the different strain values outlined above (namely, 100%, 80%, 60%, 40%, and 20%). This process was also used to measure the strain recovery of each fatigue cycle at each strain percentage. A common initial fatigue range for SMPs is five to 10 since SMPs in research show a constant shape recovery after five to 10 cycles.

To ensure that each SMP actuator sample was at room temperature prior to being stretched, the samples were submerged in the water bath, stretched, and then allowed to cool at 21.0° C. for five minutes prior to testing. For fatigue cycles greater than one, the process was repeated until the fatigue cycle number was reached.

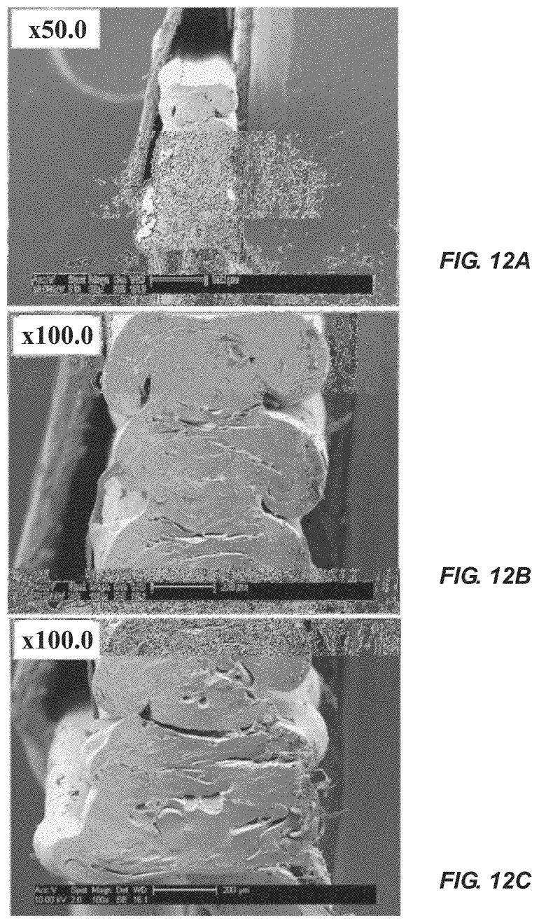

FIGS. 16A-16F are photographs each depicting multiple FFF SMP actuators 31-34 to show the progress of cyclic fatigue. FIG. 16A is a baseline (prior to any strain cycles), and FIGS. 16B-16F show the FFF SMP actuators 31-34 after undergoing first through fifth strain cycles, respectively (except FIG. 16F omits the fourth actuator 34). In each of FIGS. 16B-16F, the first (uppermost) actuator 31 was strained at 100%, the second actuator 32 was strained at 80%, and the third actuator 33 was strained at 60%. The fourth (bottom) actuator 34 in FIGS. 16B-16E was not subject to strain, to serve as a comparison for the other FFF SMP actuators 31-33. Plastic deformation and failure is seen in the FFF SMP actuators 31, 32 subjected to 100% strain and 80% strain at minimal cycles, whereas the FFF SMP actuator 33 subjected to 60% strain shows minimal polymer plastic deformation. These images suggest that 100% strain and 80% strain values exceed a suitable threshold for the tested FFF SMP actuators.

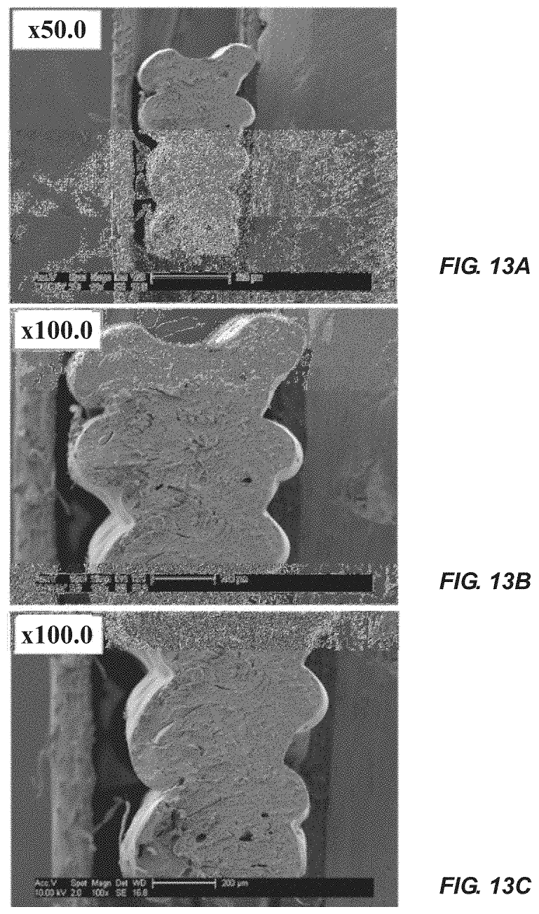

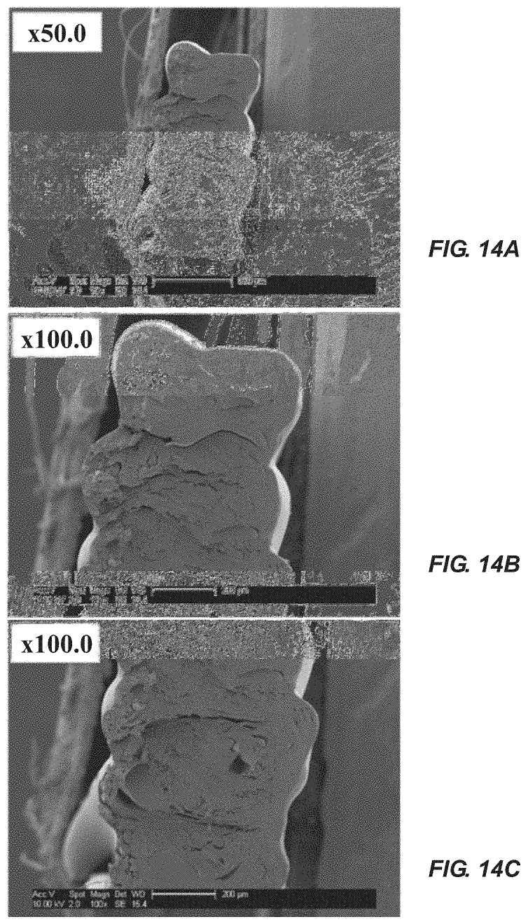

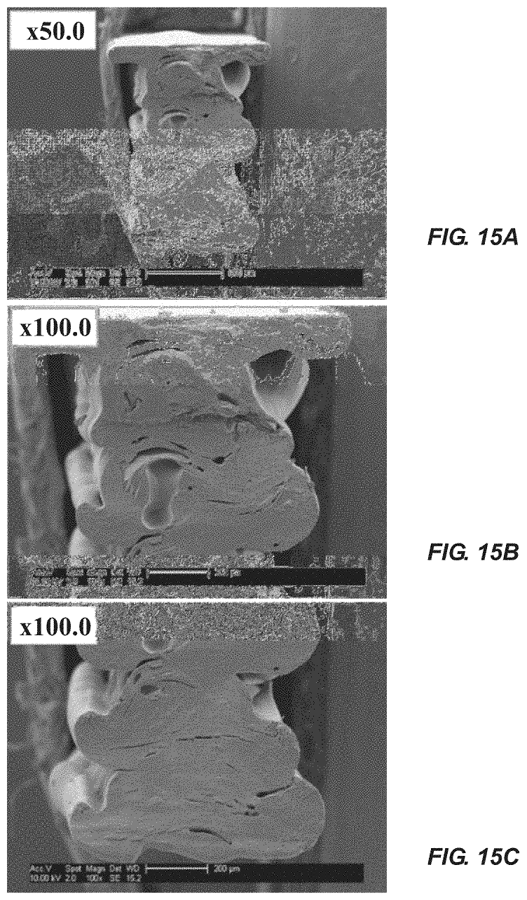

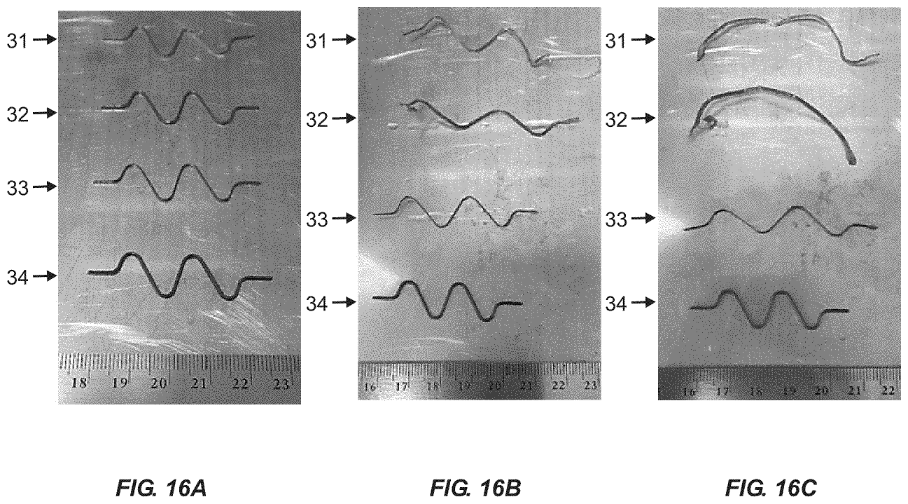

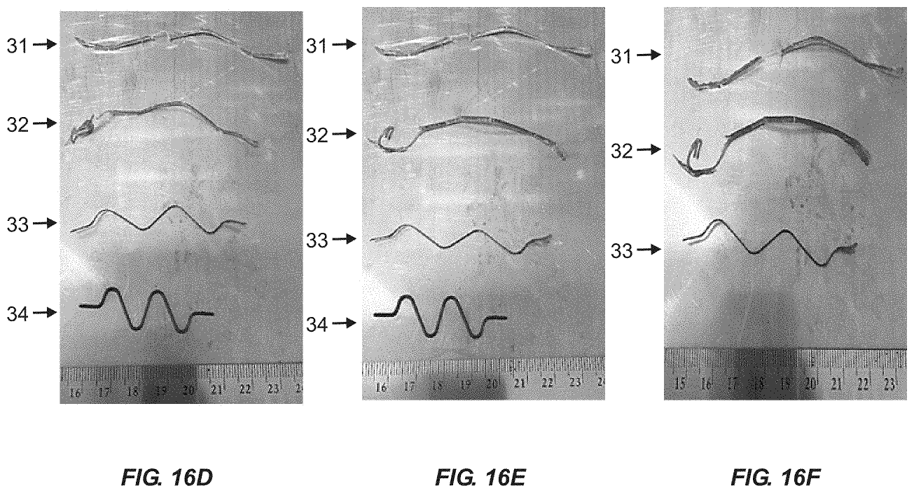

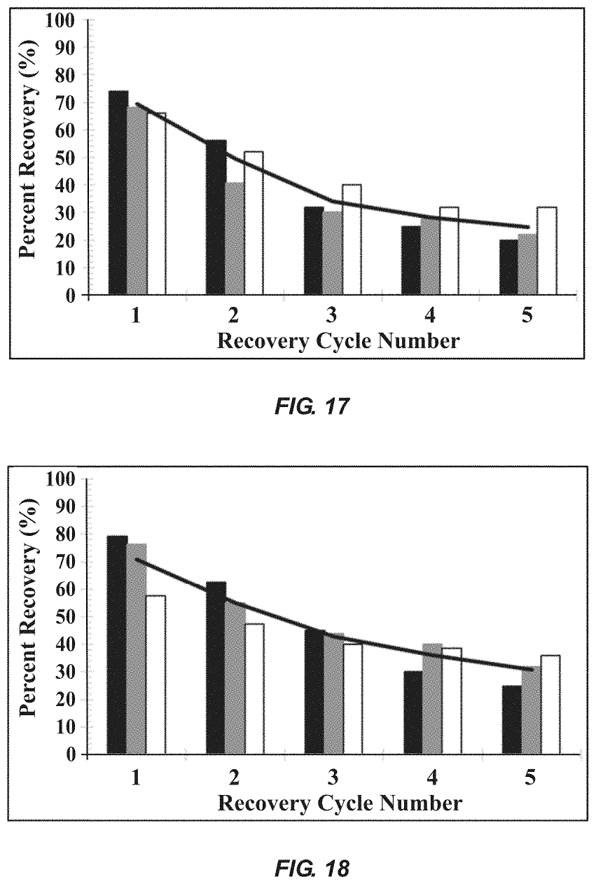

FIGS. 17-21 are bar charts of percent recovery (i.e., percent strain recovery) versus recovery cycle number for five cycles for different strain values—namely, 100% strain in FIG. 17, 80% strain in FIG. 18, 60% strain in FIG. 19, 40% strain in FIGS. 20, and 20% strain in FIG. 21. Data for multiple samples is provided in each figure, with the black line in each instance embodying an average of the tested samples for each cycle. Such charts show the shape memory effect, whereby a previously processed SMA actuator may exhibit significant (albeit incomplete) recovery of an initial size and shape after being subject to an applied stress. As is evident from FIGS. 17-21, percent of recovery tends to decline with number of cycles, but the incremental decline per cycle drops with an increasing number of cycles in a roughly asymptotic manner. Moreover, the average percent recovery for all cycles tends to decrease with increasing strain. For example, application of 100% strain in FIG. 17 results in percent recovery values in a range of from about 70% to about 30% for cycles 1-5, whereas application of 20% strain in FIG. 21 results in percent recovery values in a range of from about 80% to about 50%. FIG. 22 is a plot of average percent recovery versus strain percentage for strain percentages of 100%. 80%, 60%, 40%, and 20%, with horizontal bars representing the minimum and maximum strain values for cycles 1-5.

With continued reference to FIGS. 17-22, results after five cycles show that 100% strain has the lowest shape recovery percentage of only 24.6%; 80%-60% strain exhibit similar recoveries of 31%-29.2%, and 40-20% strain exhibit the highest shape recovery percentages of 43.2%-42.6%,. It is observed that strain values of 100%-80% result in actuator material fracture/peeling due to the increased induced stress with fatiguing. The ideal strain is 60% due to the high SR similar to the 20% for typical mammalian skeletal muscle strain, and positive material fatigue results, while 40% and 20% strain generate no stress recovery.

SMP actuator response time was determined with a FLIR One thermal imaging camera (frame rate of 0.11 seconds) and the 70° C. water bath. The SMP actuators were attached at one end a platform, allowing the other end of each actuator to be free to move or actuate, and the SMP actuators were submerged in the water bath at a low incline angle of 10-15°. Such configuration allowed for SMP actuator response to be measured without a rush of water generating high turbulence that would result if the platform was submerged parallel to the water.

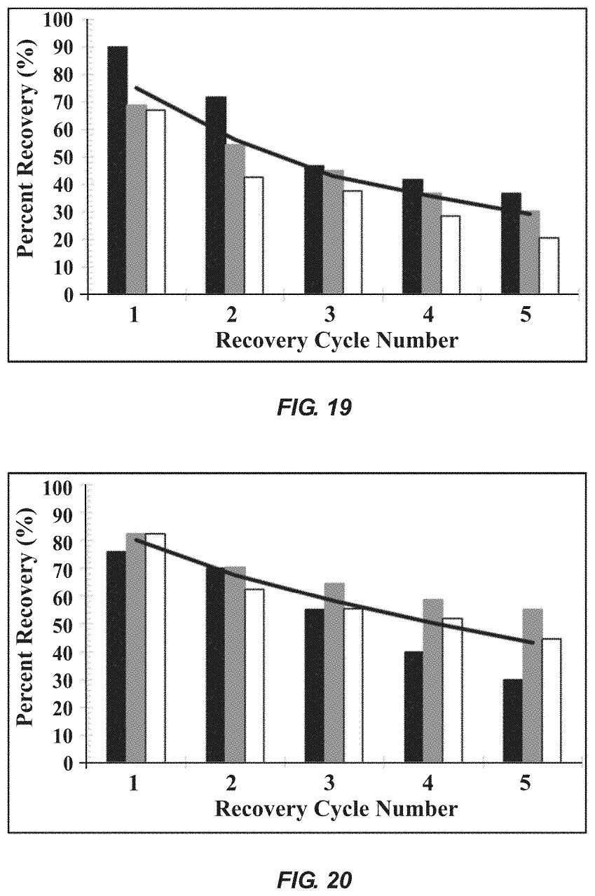

FIGS. 23A-23E are time stepped thermal images (at 0 sec, 0.33 sec, 0.66 sec, 0.77 sec, and 1.10 sec, respectively) showing the onset of shape memory effect of an SMP actuator with a layer height of 1.5 mm upon exposure to 70° C. deionized (DI) water. The black circle in FIGS. 23D and 23E shows the onset of shape recovery of the SMP actuator at 0.77 sec after contact with the 70° C. DI water. Additionally, thinner SMP actuators with thicknesses of 1.2 mm and 0.9 mm were tested for response time and exhibited response times of 0.77 seconds and 0.66 seconds, respectively. The thinner samples were not tested for contractile forces since they would not generate enough force to be measured by the force transducer on hand, and the thinner samples were difficult to remove from the bed without damage. However, testing performed on the thinner samples showed that thinner samples would exhibit an increased response time.

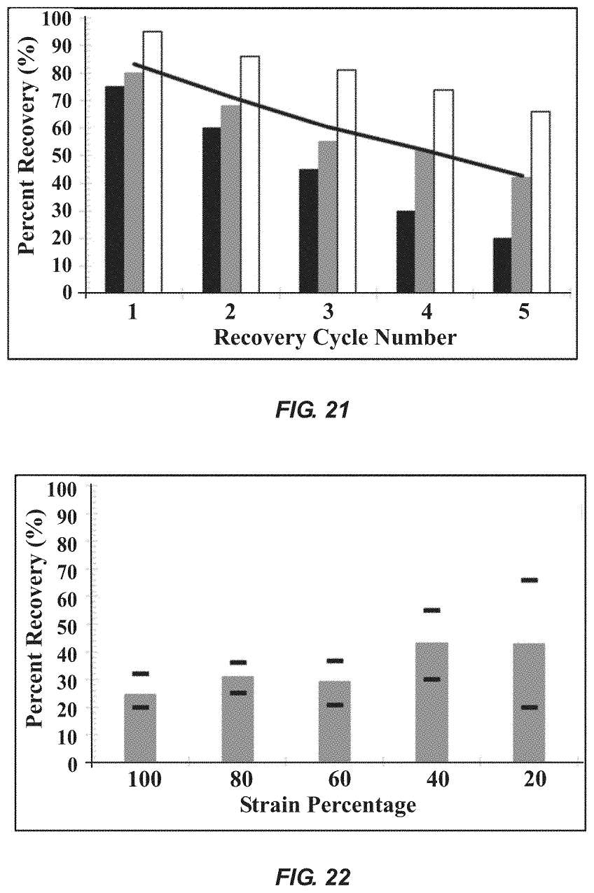

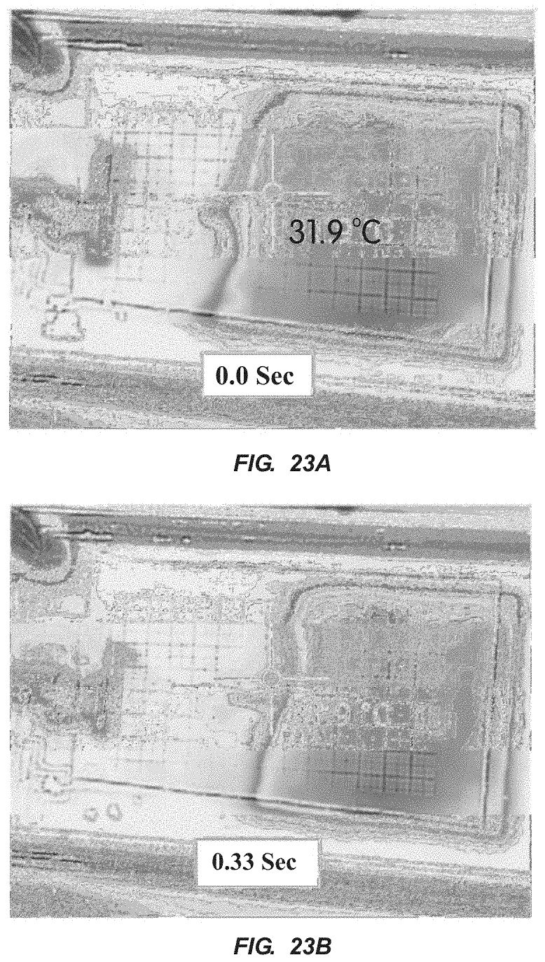

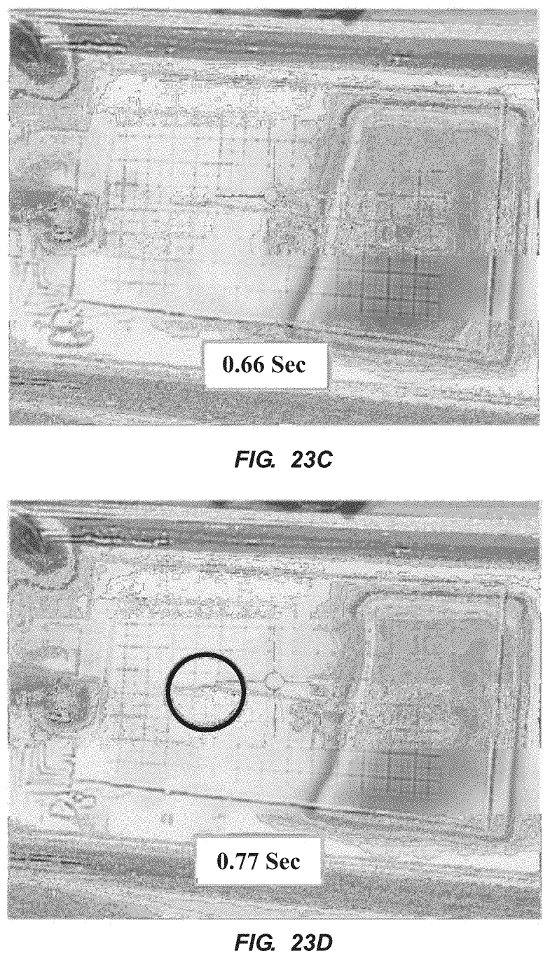

FIGS. 24-26 are line charts plotting force (N) versus temperature (degrees Celsius) for contact of SMP actuators with heated water and showing each strain for cycles 1-5, in which the SMP actuator of FIG. 24 was subjected to 100% strain, the SMP actuator of FIG. 25 was subjected to 80% strain, and the SMP actuator of FIG. 26 was subjected to 60% strain. In FIGS. 24-26, line types and colors for cycle 1-5 samples are represented as follows: thick black (cycle 1), thick gray (cycle 2), thick steel gray (cycle 3), thin black (cycle 4), and thin gray (cycle 5).

To perform actuator force testing, FFF SMP actuators were tested similar to an isometric muscle contraction, where each FFF SMP actuator was stretched to a given strain percentage, fixed at the bottom and to the force transducer, and then heated to 70° C. An isometric test setup was used since it allowed the maximum stress generated at each strain to be evaluated, and allowed for a force transducer to be employed. If a FFF SMP actuator was allowed to be displaced during contractile testing, then the FFF SMP actuator would have to lift various masses, and the generation of stress could not be recorded.

Force data was collected by placing the FFF SMP samples in an incubator with a temperature ramp rate of 1.12° C./min over a temperature range of from 30° C. to 70±5° C. to accurately show the onset and end of the contraction cycle. Additionally, an increased heating rate source was created with ceramic heating elements to demonstrate FFF SMP actuator force characteristics under a temperature ramp rate of 8.68° C./min, approximately eight times faster. The ceramic heating system was placed inside the incubator, where the incubator was heated up to 40° C. to get the entire chamber at 40° C. and then the ceramic heating elements were activated to reach 75° C. Two 40.0 mm diameter circular ceramic heating elements with an internal resistance of 4.80 were used with an applied 9.0V and 1.015 Amps to generate radiant heat.

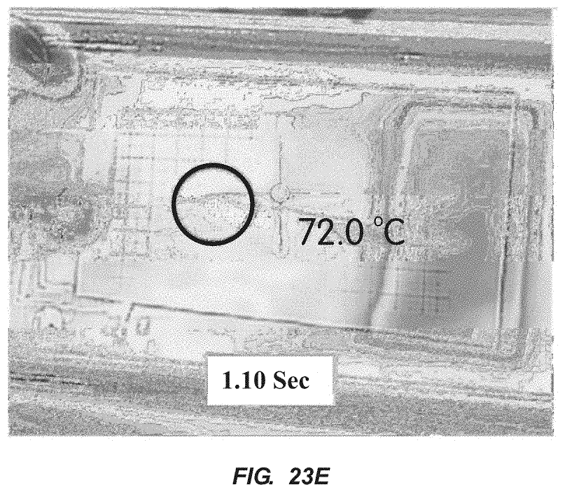

Results of the force testing are shown in FIGS. 27-36.

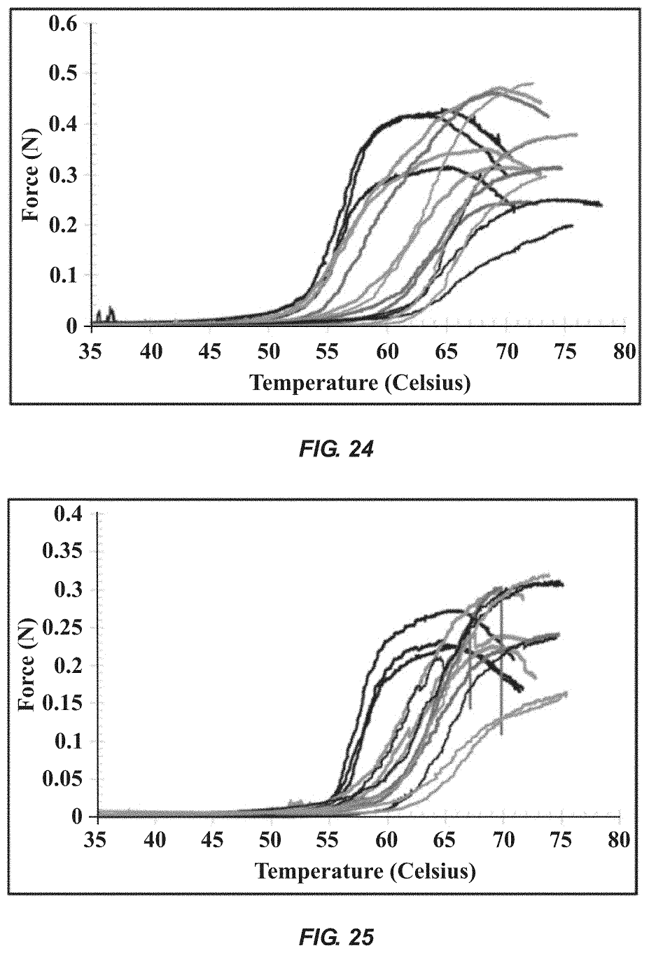

FIG. 27 is a line chart plotting force (N) versus temperature (degrees Celsius) for contact of a SMP actuator with a ceramic heater and showing each strain for cycles 1-5 with the SMP subjected to 60% strain. FIG. 28 is a line chart plotting force (N) versus temperature (degrees Celsius) for contact of SMP actuators with heated water and showing each strain for cycles 1-5 with the SMP actuators subjected to 40% strain and 20% strain, respectively.

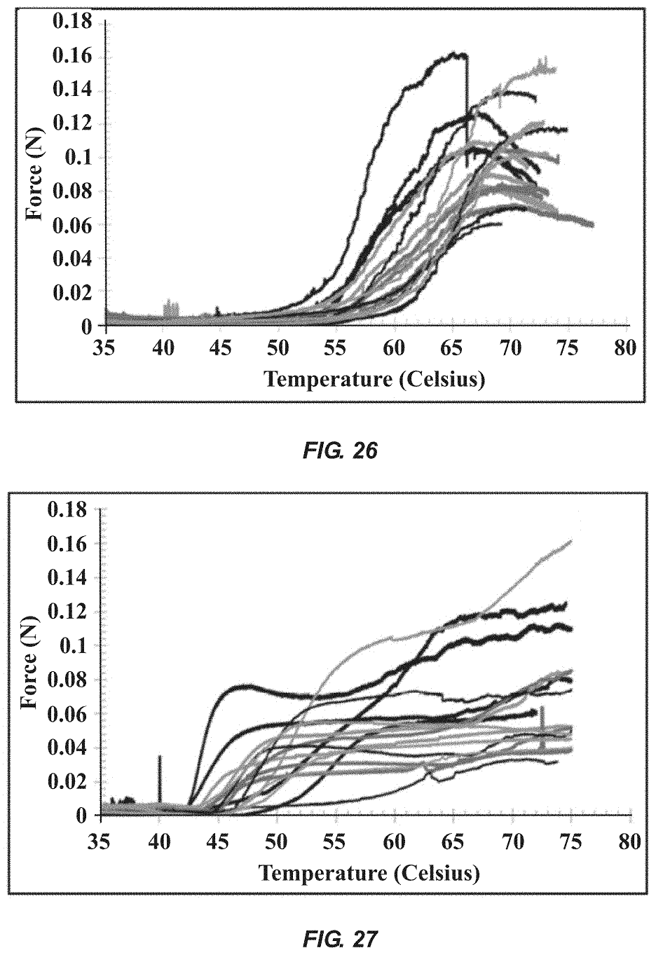

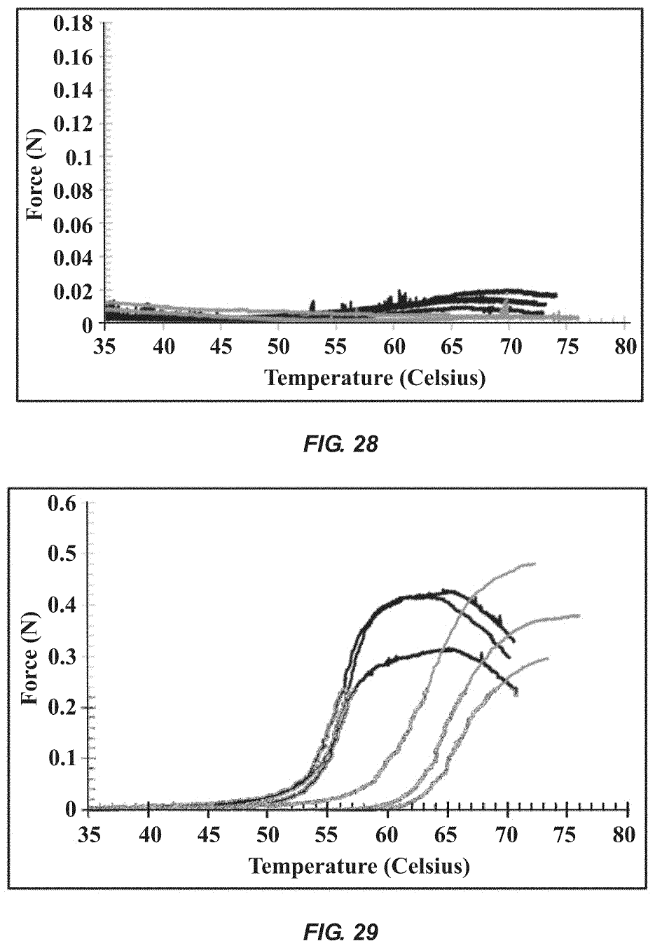

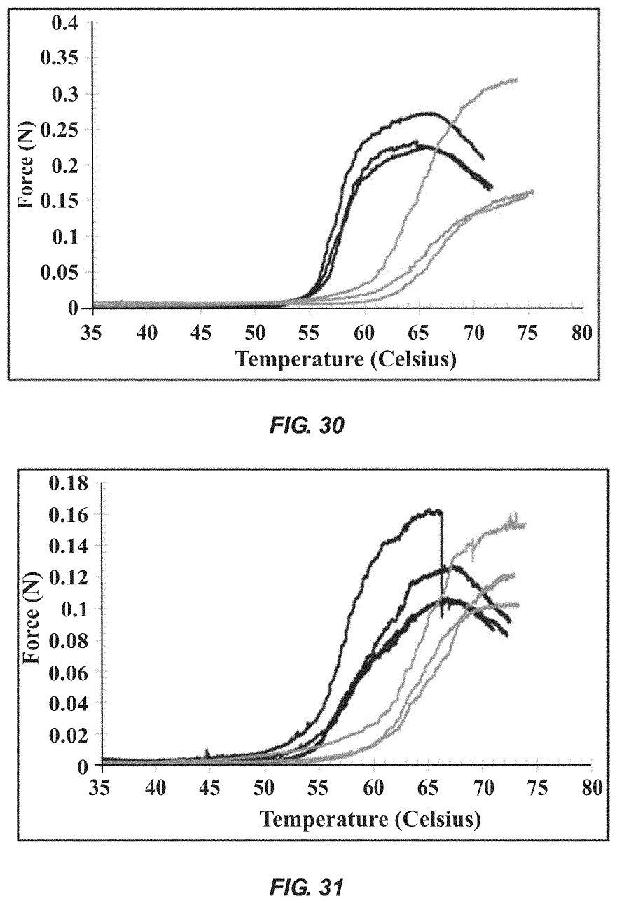

FIGS. 29-31 are line charts plotting force (N) versus temperature (degrees Celsius) for contact of SMP actuators with heated water and showing strain only for cycles 1 and 5, in which the SMP actuator of FIG. 29 was subjected to 100% strain, the SMP actuator of FIG. 30 was subjected to 80% strain, and the SMP actuator of FIG. 31 was subjected to 60% strain.

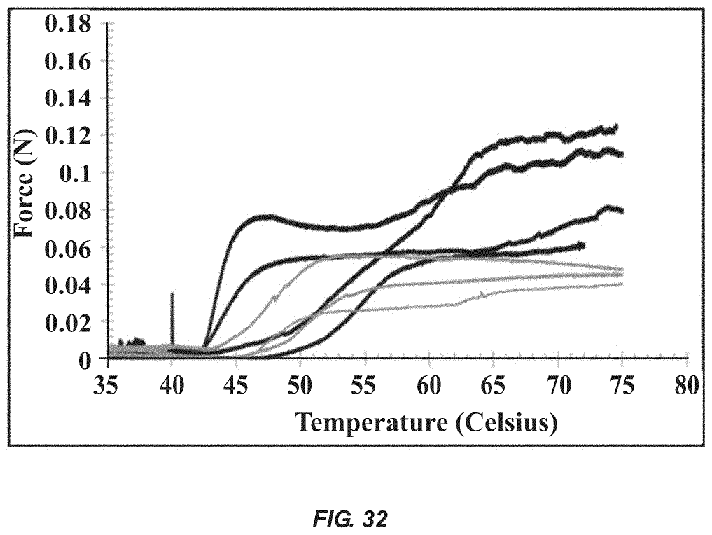

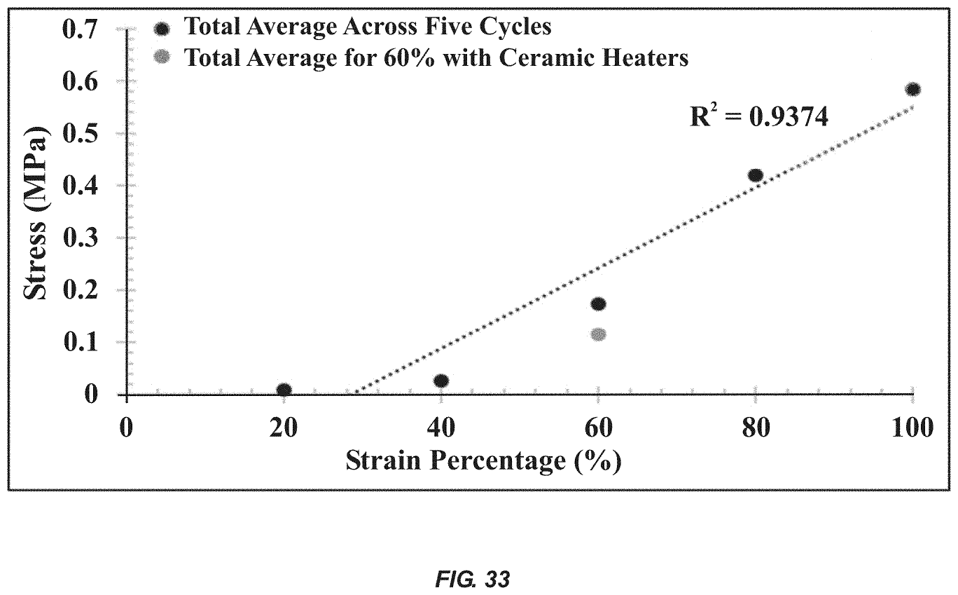

FIG. 32 is a line chart plotting force (N) versus temperature (degrees Celsius) for contact of a SMP actuator with a ceramic heater and showing strain only for cycles 1 and 5, with the SMP actuator subjected to 60% strain. FIG. 33 is a plot of average stress (MPa) versus strain percentage (%) for five cycles including contact of SMP actuators with heated water at each strain percentage of 100%, 80%, 60%, 40%, and 20%, and for five cycles including contact of a SMP actuator with a ceramic heater at 60% strain.

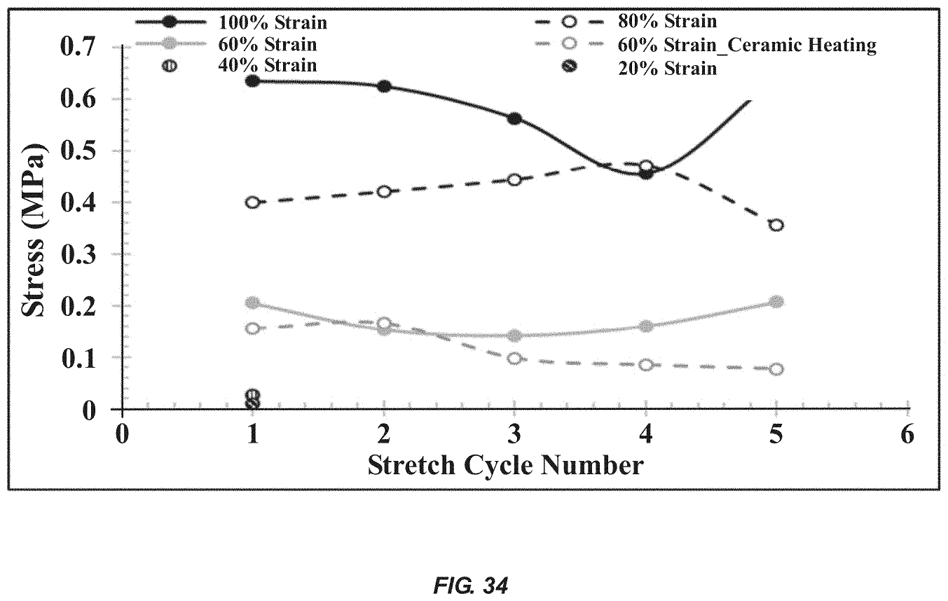

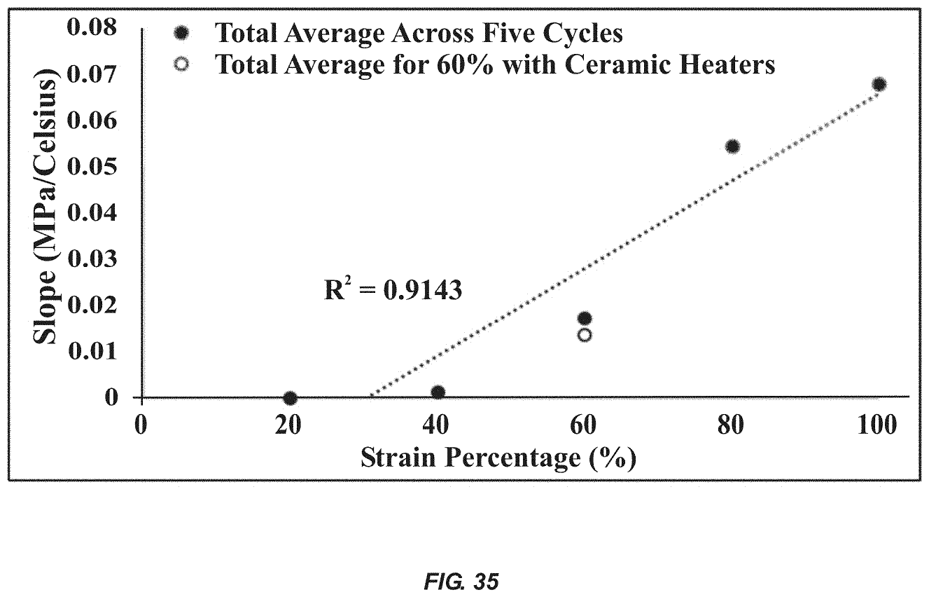

FIG. 34 is a plot of average stress (MPa) versus stretch cycle number for strain percentage (%) for five cycles including contact of SMP actuators with heated water at each strain percentage of 100%, 80%, 60%, 40%, and 20%, and for five cycles including contact of a SMP actuator with a ceramic heater at 60% strain. FIG. 35 is a plot of average slope (MPa/degree Celsius) versus strain percentage (%) for five cycles including contact of SMP actuators with heated water at each strain percentage of 100%, 80%, 60%, 40%, and 20%, and for five cycles including contact of a SMP actuator with a ceramic heater at 60% strain.

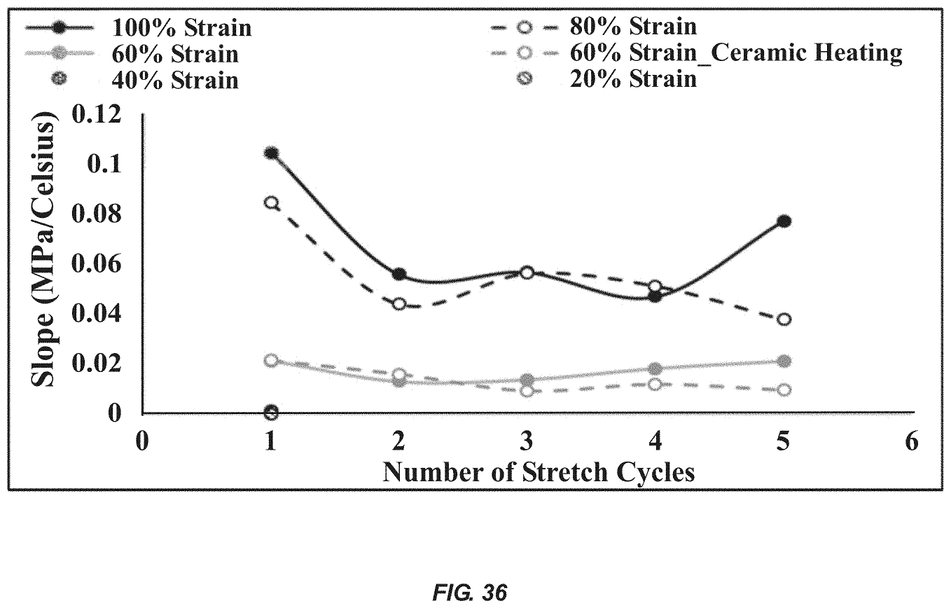

FIG. 36 is a plot of slope (MPa/degree Celsius) versus stretch cycle number for strain percentage (%) for five cycles including contact of SMP actuators with heated water at each strain percentage of 100%, 80%, 60%, 40%, and 20%, and for five cycles including contact of a SMP actuator with a ceramic heater at 60% strain.

From the results shown in FIGS. 27-36, the FFF SMP actuator samples with 100% strain cycled one to five times showed the largest peak force along with the greatest rate/slope of force generation. The force and rate decreased linearly as the strain percentage decreased until 40% and 20% when the SMP actuator could not generate enough internal stress to cause a recovery stress/force. The peak contraction values for each strain were comparable to mammalian skeletal muscle contractions and followed a non-linear force generation due to the spring shape design and force generation throughout the entire glass transition zone.

Where mammalian contraction values range from 0.1 MPa (typical/average) to 0.45 MPa (maximum), SMP actuators characterized herein exhibit contraction values ranging from 0.58 MPa to 0.17 MPa for 100% to 60% strain, respectively. It is observed that after cycling the SMP actuator once, there is an initial drop in peak force and rate averages, followed by a relative leveling or constant peak force and rate. Additionally, it is apparent that the 60% strain SMP actuator samples tested in the ceramic heating system show comparable result averages to counterparts tested in the incubator chamber. This demonstrates that increasing the heating rate will not affect the performance of a FFF SMP actuator. 60% strain was chosen as a preferred value since SMP actuator samples strained to this value demonstrated the highest strain recovery with minimal material failure.

The functionality of FFF SMP actuators disclosed herein was demonstrated with contractile, shape recovery, and passive actuator tensile testing at a loading rate of 50 mm/min. Shape recovery, along with initial response time, show that among the strain values tested, FFF SMP actuators with 60% strain are optimal for shape recovery with a response time of 0.77 sec.

Since cyclic fatiguing appears to level off after five cycles for all strains, 60% strain is ideal since it achieves 29.2% recovery with minimal plastic deformation.

Strains of 80% and 100% exhibit higher peak force and higher response rates, but due to the increased applied stress, FFF SMP actuators subjected to these strain values have lower shape recovery due to plastic deformation/breakage. Since the FFF SMP actuator according to certain embodiments has an overall length of 50 mm with 78 mm in material length, when the material is strained well past 78 mm it begins to fail. A 60% strain value for a SMP actuator having an initial overall length of 78 mm corresponds to displacement to 80 mm, which results in minimal applied strain (2.0 mm) to the 78 mm material length after full stretch while still generating internal stress. This allows the FFF actuator to stay within the elastic region of the composite, as shown in the stress-strain plot of FIG. 3.

The spring shape design allows a FFF SMP actuators disclosed herein to have a high deformation prior to change in cross-section, and high shape recovery response initially due to the decrease in cross section (corresponding to high stress on the polymer-chain structure) followed by a lower response once the cross section is recovered (corresponding to low stress on the polymer-chain structure). In contrast, a straight line/wire would only allow for deformation of the polymer with a decrease in cross-section, which would cause plastic deformation at low strain levels due to the brittle nature of the polymer and decrease shape recovery. This effect is seen where the 100%-80% strain samples exhibit a low shape recovery due to plastic deformation, while the 60%-20% strain samples exhibit a high shape recovery due to the lack of plastic deformation.

Results from the force/contractile testing shown in FIGS. 33-36 demonstrated that as the strain percentage decreases, the peak force and rate (stress/° C.) decreased as a linear function. This linear decrease is due to the function of input/applied stress generated during stretching and the amount of stretching/displacement. As the strain decreased, the applied stress decreased since there was less elongation along the cross-section of a FFF SMP actuator.

Therefore, when using strains of 40% and 20%, no elongation or decrease in the cross-section was exhibited after stretching. This is observed since the strain values higher than 40% showed consistent force generation, while 40% and 20% strain values showed little to zero force generation.

With continued reference to FIGS. 33-36, fatigue cycling of the FFF SMP actuator samples showed an initial decrease in peak force and rate after one cycle, followed by a relatively constant peak force and rate. The onset of force generation was shifted with each cycle, with the largest offset happening after the first cycle. Additionally, the force generation followed a non-linear pattern of contractile force due to the gradient response to thermal input and shape recovery, and SMP actuator spring shape. The SMP actuators responded non-linearly, since force generation was initiated prior to the Tg, near 50° C., and continued through the Tg range, near 75° C., along with the spring shape allowing a dual shape recovery effect as described above.

FFF SMP actuators according to certain embodiments described herein exhibit non-linear contractile properties that provide peak contractions similar to mammalian muscles, ranging from 0.58 MPa to 0.17M Pa for 100% to 60% strain respectively. Additionally, the ideal strain of 60% has a strain recovery of 29.2%, which is comparable to the 20% for typical mammalian skeletal muscle strain.

In certain embodiments, multiple SMP actuators (e.g., FFF SMP actuators as disclosed herein) may be utilized in a joint of a prosthetic device, with certain actuators utilized as agonists and other actuators utilized as antagonists.

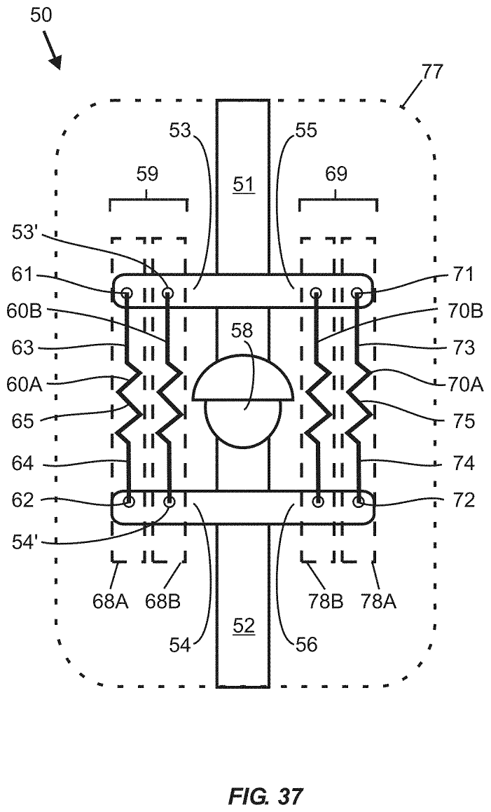

FIG. 37 is a schematic illustration of a prosthetic device 50 including a plurality of thermally responsive shape memory actuators (optionally embodying FFF SMP actuators as disclosed herein) 60A, 60B, 70A, 70B, a movable joint 58 connected between first and second structural members 51, 52, and multiple anchors 53′, 54′ associated with the structural members 51, 52. The four thermally responsive shape memory actuators 60A, 60B, 70A, 70B may be identical in character, but only the outermost two shape memory actuators 60A, 70A are described in detail hereinafter. Each outermost thermally responsive shape memory actuator 60A, 70A comprises a body including a non-linear segment 65, 75 disposed between two linear segments 63, 64, 73, 74 bounded by body ends 61, 62, 71, 72, and the body preferably comprises a shape memory polymer material. The movable joint 58 is configured to permit pivotal movement between the first structural member 51 and the second structural member 52. A first anchor element 53 and a second anchor element 55 are associated with the first structural member 51, and a third anchor element 54 and a fourth anchor element 56 are associated with the second structural member 52. A first group of thermally responsive shape memory actuators 59 (namely, actuators 60A, 60B) is coupled between the first anchor element 53 and the third anchor element 54, and is configured to promote pivotal movement between the first structural member 51 and the second structural member 52 in a first direction. Additionally, a second group of thermally responsive shape memory actuators 69 (namely, actuators 70A, 70B) is coupled between the second anchor element 55 and the fourth anchor element 56, and is configured to promote pivotal movement between the first structural member 51 and the second structural member 52 in a second direction that differs from the first direction. Optionally, a flexible tube or other jacket 68A, 68B, 78A, 78B may be formed around each

SMP actuator 60A, 60B, 70A, 70B to selectively circulate warm fluid (e.g., coupled with a pump and thermal source, not shown) to permit the SMP actuators 60A, 60B, 70A, 70B to be actuated when needed. Additionally, the prosthetic device 50 may include an outer tube or other covering element 77 that contains or otherwise covers the first and second structural members 51, 52, the movable joint 58, the anchor elements 53-56, and the groups of shape memory actuators 59, 69.

Upon reading the following description in light of the accompanying drawing figures, those skilled in the art will understand the concepts of the disclosure and will recognize applications of these concepts not particularly addressed herein. Those skilled in the art will recognize improvements and modifications to the preferred embodiments of the present disclosure. All such improvements and modifications are considered within the scope of the concepts disclosed herein and the claims that follow.

What is claimed is:

1. A prosthetic device comprising:
a plurality of thermally responsive shape memory actuators, wherein each thermally responsive shape memory actuator of the plurality of thermally responsive shape memory actuators comprises a body including a non-linear segment disposed between two body ends, and the body comprises a shape memory polymer material;
a first structural member, a second structural member, and a movable joint connected between the first structural member and the second structural member, wherein the movable joint is configured to permit pivotal movement between the first structural member and the second structural member;
a first anchor element and a second anchor element associated with the first structural member;
a third anchor element and a fourth anchor element associated with the second structural member;
wherein at least one first thermally responsive shape memory actuator of the plurality of thermally responsive shape memory actuators is coupled between the first anchor element and the third anchor element, and is configured to promote pivotal movement between the first structural member and the second structural member in a first direction; and
wherein at least one second thermally responsive shape memory actuator of the plurality of thermally responsive shape memory actuators is coupled between the second anchor element and the fourth anchor element, and is configured to promote pivotal movement between the first structural member and the second structural member in a second direction that differs from the first direction.

2. The prosthetic device of claim 1, wherein the body of each thermally responsive shape memory actuator of the plurality of thermally responsive shape memory actuators comprises a plurality of fused shape memory polymer elements comprising a plurality of dots, rods, or layers.

3. The prosthetic device of claim 1, wherein the body of each thermally responsive shape memory actuator of the plurality of thermally responsive shape memory actuators is produced by a fused filament fabrication process.

4. The prosthetic device of claim 1, wherein for each thermally responsive shape memory actuator of the plurality of thermally responsive shape memory actuators, the body includes a first end, a second end, and at least one non-linear segment disposed between the first end and the second end.

5. The prosthetic device of claim 4, wherein for each thermally responsive shape memory actuator of the plurality of thermally responsive shape memory actuators, the body comprises a first substantially straight segment proximate to the first end and a second substantially straight segment proximate to the second end, with the at least one non-linear segment being arranged between the first substantially straight segment and the second substantially straight segment.

6. The prosthetic device of claim 1, wherein for each thermally responsive shape memory actuator of the plurality of thermally responsive shape memory actuators, the shape memory polymer material of the body comprises a linear aliphatic thermoplastic polyester and at least one other polymer.

7. The prosthetic device of claim 6, wherein the linear aliphatic thermoplastic polyester comprises at least one of a poly(L-lactide)-based polymer or a poly(ε-caprolactone)-based polymer.

8. The prosthetic device of claim 6, wherein the shape memory polymer material of the body comprises (i) poly(L-lactide) or poly(ε-caprolactone) and (ii) at least one other polymer.

9. The prosthetic device of claim 1, wherein the body of each thermally responsive shape memory actuator of the plurality of thermally responsive shape memory actuators is pre-strained by heating and elongation in a range of 140% to 170% of an initial length of the body.

10. The prosthetic device of claim 1, wherein:
the at least one first thermally responsive shape memory actuator comprises a plurality of first thermally responsive shape memory actuators; and
the at least one second thermally responsive shape memory actuator comprises a plurality of second thermally responsive shape memory actuators.

11. The prosthetic device of claim 1, wherein:
at least one first flexible tube or jacket is provided around the at least one first thermally responsive shape memory actuator; and
at least one first flexible tube or jacket is provided around the at least one first thermally responsive shape memory actuator.

12. The prosthetic device of claim 1, further comprising an outer tube or covering element containing the plurality of thermally responsive shape memory actuators, the first and second structural members, the movable joint, and the first through fourth anchor elements.

13. The prosthetic device of claim 1, further comprising a pump and a thermal source, wherein the prosthetic device is configured to circulate warm fluid through one or more of the at least one first flexible tube or jacket and the at least one second flexible tube or jacket.
